SEQUENCE LISTING (21 sequences)

SEQ ID NO: 1 - protein, 5 aa, artificial
  CDR1 = hypervariable region 1 of heavy chain of AIN457
NYWMN

SEQ ID NO: 2 - protein, 17 aa, ARTIFICIAL
  CDR2 = hypervariable region 2 of heavy chain of AIN457
AINQDGSEKYYVGSVKG

SEQ ID NO: 3 - protein, 18 aa, ARTIFICIAL
  CDR3 = hypervariable region 3 of heavy chain of AIN457
DYYDILTDYYIHYWYFDL

SEQ ID NO: 4 - protein, 12 aa, ARTIFICIAL
  CDR1′ = hypervariable region 1 of light chain of AIN457
RASQSVSSSYLA

SEQ ID NO: 5 - protein, 7 aa, ARTIFICIAL
  CDR2′ = hypervariable region 2 of light chain AIN457
GASSRAT

SEQ ID NO: 6 - protein, 9 aa, ARTIFICIAL
  CDR3′ = hypervariable region 3 of light chain AIN457
QQYGSSPCT

SEQ ID NO: 7 - DNA, 381 bp, HOMO SAPIENS
  CDS (1)..(381)
gaggtgcagttggtggagtctgggggaggcttggtccagcctggggggtccctgagactctcctgtgcagcctctggattcacctttagtaactattggatgaactgggtccgccaggctccagggaaagggctggagtgggtggccgccataaaccaagatggaagtgagaaatactatgtgggctctgtgaagggccgattcaccatctccagagacaacgccaagaactcactgtatctgcaaatgaacagcctgagagtcgaggacacggctgtgtattactgtgtgagggactattacgatattttgaccgattattacatccactattggtacttcgatctctggggccgtggcaccctggtcactgtctcctca

SEQ ID NO: 8 - protein, 127 aa, HOMO SAPIENS [sequence omitted]

SEQ ID NO: 9 - DNA, 327 bp, HOMO SAPIENS
  CDS (1)..(327)
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtgcaccttcggccaagggacacgactggagattaaacga

SEQ ID NO: 10 - protein, 109 aa, HOMO SAPIENS
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKR

SEQ ID NO: 11 - protein, 10 aa, artificial
  CDR1-x = hypervariable domain x of heavy chain of AIN457
GFTFSNYWMN

SEQ ID NO: 12 - protein, 11 aa, artificial
  CDR2-x = hypervariable domain of heavy chain x of AIN457
AINQDGSEKYY

SEQ ID NO: 13 - protein, 23 aa, ARTIFICIAL
  CDR3-x = hypervariable domain x of heavy chain AIN457
CVRDYYDILTDYYIHYWYFDLWG

SEQ ID NO: 14 - protein, 215 aa, homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 457 aa, homo sapiens
EVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 7 aa, artificial
  IMGT LCDR1
QSVSSSY

SEQ ID NO: 17 - protein, 3 aa, artificial
  IMGT LCDR2
GAS

SEQ ID NO: 18 - protein, 9 aa, Artificial
  IMGT LCDR3
QQYGSSPCT

SEQ ID NO: 19 - protein, 8 aa, artificial
  IMGT HCDR1
GFTFSNYW

SEQ ID NO: 20 - protein, 8 aa, Artificial
  IMGT HCDR2
INQDGSEK

SEQ ID NO: 21 - protein, 20 aa, Artificial [sequence omitted]

(19) United States
(12) Patent Application Publication
(10) Pub. No.: US 2023/0009657 A1
(43) Pub. Date: Jan. 12, 2023

(54) METHODS OF TREATING LUPUS NEPHRITIS USING INTERLEUKIN-17 (IL-17) ANTAGONISTS

(71) Applicant: NOVARTIS AG, Basel (CH)

(72) Inventors: Wolfgang HUEBER, Basel (CH); Shephard MPOFU, Oberwil (CH); Luminita PRICOP, New York, NY (US)

(21) Appl. No.: 17/777,188

(22) PCT Filed: Nov. 17, 2020
(86) PCT No.: PCT/IB2020/060796

§ 371 Date: May 16, 2022

Related U.S. Application Data
(60) Provisional application No. 62/937,482, filed on Nov. 19, 2019.

(51) Int. Cl. C07K 16/24; A61P 13/12

(52) U.S. Cl. CPC C07K 16/244; A61P 13/12; A61K 2039/54

(57) ABSTRACT

The present disclosure relates to methods for treating Lupus Nephritis (LN) using IL-17 antagonists, e.g., secukinumab. Also disclosed herein are IL-17 antagonists, e.g., IL-17 antibodies, such as secukinumab, for treating LN patients, as well as medicaments, dosing regimens, pharmaceutical formulations, dosage forms, and kits for use in the disclosed uses and methods.

### TECHNICAL FIELD

The present disclosure relates to methods for treating lupus nephritis (LN) using IL-17 antagonists, e.g., IL-17 antibodies, e.g., secukinumab.

### BACKGROUND OF THE DISCLOSURE

LN represents inflammation of the kidneys and is one of the organ-specific disease manifestations of Systemic Lupus Erythematosus (SLE) (Waldman and Madaio (2005) Lupus 14(1):19-24). LN is a chronic inflammatory disease characterized by auto-antibody production and other distinct immunological abnormalities (Gurevitz et al. (2013) Consult Pharm 28: 110-21). It is categorized histologically into six classes by the International Society of Nephrology/Renal Pathology Society (ISN/RPS) classification system that has become the standard for renal biopsy interpretation because of improved correlation with prognostic and therapeutic outcomes. (Weening et al. (2004) J Am Soc Nephrol. 15(2):241-50; Markowitz et al. (2007) Kidney Int; 71(6):491-5). Immune complex formation in LN is the result of systemic autoimmunity and is a hallmark of the disease (Waldman (2005) Lupus 14(1):19-24; Nowling (2011) Arthritis Res Ther. 13(6):250). Once formed, immune complexes activate complement, which can injure renal cells, leading to either mesangial LN (class I, II), endothelial-proliferative LN (class III, IV), or nephrotic syndrome (class V).

The pathogenesis of LN is complex and involves both the innate and adaptive immune system, various cytokines and tissue, and immune cells. Intra-renal inflammation is maintained via local cytokine and chemokine production and by cells of the innate immune system, such as neutrophils, that are attracted into the glomerulus and interstitium. Targeting local release of pro-inflammatory cytokines by blocking individual cytokines, may enhance treatment efficacy in autoimmunity without increasing systemic immunosuppression. (Allam (2008) Curr Opin Rheumatol; 20(5):538-44; Yu et al. (2017) Nat Rev Nephrol; 13(8):483-95).

Despite recent advances in treatment for several autoimmune diseases, there is still no adequate treatment for LN. It remains a major cause of morbidity and mortality, with 22% of LN patients progressing to ESRD within 15 years (Faurschou et al. (2010) Arthritis Care & Research 62(6):873-80; Tektonidou et al. (2016) Arthritis Rheumatol. 68(6):1432-41). There are currently no specific FDA-approved therapies for LN. Current treatments are non-specific, aimed at slowing progression with general immunosuppression. Renal response rates remain suboptimal, underscoring the persistent high unmet need in the treatment of patients with LN.

The American College of Rheumatology (ACR) Guidelines for Screening, Treatment, and Management of Lupus Nephritis have been published in 2012, and are recognized internationally (Hahn et al. (2012) Arthritis Care Res (Hoboken); 64:797-808]. The Joint European League Against Rheumatism/European Renal Association—European Dialysis and Transplant Association (EULAR/ERA-EDTA) guideline has been released the same year (Bertsias et al. (2012) Ann Rheum Dis. 71:1771-82). While there is general agreement of the treatments recommended in these guidelines, these medications have not received either US or European regulatory agency approvals for the indication of LN. It is recommended that LN patients receive several adjunctive medications, such as hydroxychloroquine (HCQ), a lipid-lowering statin and renin-angiotensin-aldosterone system inhibitors (ACE/ARB inhibitors). Where indicated for symptomatic manifestations, steroids are the mainstay of treatment for Class I minimal change LN disease. The ACR guideline does not recommend additional immunosuppression for class II LN. The EULAR/ERA-EDTA guideline recommends low to moderate doses of oral glucocorticoids alone or in combination with azathioprine in cases of proteinuria and hematuria.

The guidelines are uniform in their recommendations for therapy for class III and IV LN and include a sequence of induction and maintenance phases. For patients with class III or IV proliferative glomerulonephritis, the ACR guidelines agree on induction therapy with mycophenolate mofetil (MMF) or i.v. cyclophosphamide (CYC), with or without initial pulses of i.v. methylprednisolone. With current induction regimens, <60% of class III to V patients achieve a complete response (Appel et al. (2009) J Am Soc Nephrol. 20: 1103-1112). Among those who attain a complete renal response (CRR) with current standard-of-care (SoC), nearly half of the patients had a relapse. The rate of relapse in these patients was 5 to 15 per 100 patient-years (Grootscholtenet al. (2006) Nephrol Dial Transplant 21:1465-1469).

Patients with class V lupus nephritis are typically treated with antiproteinuric and antihypertensive medications and can receive corticosteroids and immunosuppressive therapy as required depending on the presence of persistent nephrotic proteinuria.

Several histological features affect treatment decisions and prognosis. For example, patients with high “activity” (A) lesions are typically treated with immunosuppression, whereas those with “chronic” (C) lesions may not receive immunosuppressive therapy because of a poorer response prognosis (Hiramatsu et al. (2008) Rheumatology (Oxford) 47:702-07].

Medical treatment of LN with the current SoC achieves a satisfactory renal response only in about half of the patients, and carries a significant burden with respect to safety. Non-responders to the current induction and maintenance therapies have the worst outcomes. Among patients with class IV LN, about 40% developing ESRD at 15 years (Tektonidou et al. (2016) Arthritis Rheumatol. 68(6):1432-41). Thus, despite the aggressive nature of SoC treatment, only up to 40% of patients achieve a CRR after 1 year (Rovin et al. (2014) Am J Kidney Dis. 63(4):677-90). In addition, current LN treatment regimens have substantial side effects from glucocorticoids and prolonged immunosuppression (Schwartz et al. (2014) Curr Opin Rheumatol. 26:502-09). Immunosuppressed LN patients are at significant risk of developing serious infections. In a multiethnic Medicaid cohort, the incidence rate of serious infections was >2-fold higher in LN than SLE patients (Feldman et al. (2015) Arthritis Rheumatol. 67:1577-85).

Given the severity of the condition and the lack of approved therapy, there is a high unmet medical need for safe and effective long-term therapies (i.e., stand alone or as add-on therapies) for the treatment of LN.

### SUMMARY OF THE DISCLOSURE

IL-17A and Th17 cells may play roles in the pathogenesis of LN, contributing to the glomerular injury and the persistence of inflammation and renal damage (Zhang et al. (2009) J Immunol. 183(5):3160-9; Crispin et al. (2008) J Immunol. 181:8761-66). High levels of IL-17 predict poor histopathological outcome after immunosuppressive therapy in patients with LN (Zickert et al. (2015) BMC Immunol. 16:7). A subset of T-cells infiltrate the kidneys of patients with LN and represent the major source for IL-17 (Crispin et al. (2008), supra). IL-17 has a potential to induce the production of additional inflammatory cytokines and chemokines and to promote recruitment of inflammatory cells such as monocytes and neutrophils to inflamed organs. Higher levels of glomerular IL-17 and IL-23 expression are observed in renal biopsies from class IV LN patients as compared with those from minimal change nephropathy patients and normal controls. Both glomerular IL-17 and IL-23 expression levels positively correlate with renal histological activity index for LN patients (Chen et al. (2012) Lupus 21:1385). The urinary expression of Th17-related genes, including ILI 7 and IL23, is increased and associated with the activity of LN (Kwan et al. (2009) Rheumatology (Oxford) 48(12):1491-7).

Secukinumab (see, e.g., WO2006/013107 and WO2007/117749) has a very high affinity for IL-17, i.e., a K_{D} of about 100-200 pM and an IC₅₀ for in vitro neutralization of the biological activity of about 0.67 nM human IL-17A of about 0.4 nM. Thus, secukinumab inhibits antigen at a molar ratio of about 1:1. This high binding affinity makes the secukinumab antibody particularly suitable for therapeutic applications. Furthermore, secukinumab has a long half-life, i.e., about 4 weeks, which allows for prolonged periods between administration, an exceptional property when treating chronic life-long disorders, such as LN.

A recent case study reports the successful treatment of a patient with coexisting SLE and axials spondyloarthritis using 150 mg secukinumab weekly for 4/52 weeks, followed by monthly administration thereafter (Ecclestone et al. (2019) Abst. 109; Rheumatology, 58:3, kez108.017) However, urinalysis was normal in this patient, suggesting the patient did not have LN. A case study of a patient having refractory LN (refractory to both MMF and cyclophosphamide therapy) and concomitant psoriasis vulgaris suggests that treatment with secukinumab may have contributed to the improvement in renal function and decrease in urine protein levels in this patient (Satoh et al. (2018) Lupus 27(7):1202-06). The patient in Satoh et al. was treated with initial doses of 300 mg secukinumab, followed by later monthly doses of 150 mg secukinumab. The total length of secukinumab treatment is not reported in Satoh et al., and hence the long-term safety of the secukinumab regimen used by the clinicians in Satoh et al. cannot be assessed.

We have now devised novel treatments for LN patients (in particular, LN patients already receiving standard-of-care [SoC] LN treatments, e.g., patients receiving MMF [or CYC] with or without corticosteroids) with IL-17 antagonists, e.g., IL-17 antibodies or antigen-binding fragments thereof, e.g., secukinumab, that are safe, effective and provide sustained responses for patients. Importantly, because current SoC treatments for LN have strong immunosuppressive effects, any add-on therapy must maintain a favorable risk/benefit profile. Hence, these novel treatments satisfy a long-felt need of clinicians and patients for a safe, sustained, and effective therapy (particularly an add-on therapy) for LN.

Disclosed herein are methods of treating LN, comprising subcutaneously (SC) administering to a patient in need thereof a dose of about 150 mg (e.g., 150 mg) of an IL-17 antibody, or an antigen-binding fragment thereof (e.g., secukinumab), weekly during weeks 0, 1, 2, 3, and 4, and thereafter administering a SC dose of about 150 mg (e.g., 150 mg) of the IL-17 antibody, or an antigen-binding fragment thereof (e.g., secukinumab) every four weeks.

Disclosed herein are methods of treating LN, comprising subcutaneously (SC) administering to a patient in need thereof a dose of about 300 mg (e.g., 300 mg) of an IL-17 antibody, or an antigen-binding fragment thereof (e.g., secukinumab), weekly during weeks 0, 1, 2, 3, and 4, and thereafter administering a SC dose of about 300 mg (e.g., 300 mg) of the IL-17 antibody, or an antigen-binding fragment thereof (e.g., secukinumab) every four weeks.

Disclosed herein are also methods of treating LN, comprising intravenously (IV) administering to a patient in need thereof a dose of about 4 mg/kg—about 9 mg/kg (preferably about 6 mg/kg) of an IL-17 antibody, or an antigen-binding fragment thereof (e.g., secukinumab) once during week 0, and thereafter administering an IV dose of about 2—about 4 mg/kg (preferably about 3 mg/kg) of the IL-17 antibody, or an antigen-binding fragment thereof (e.g., secukinumab) every 4 weeks (monthly), beginning during week 4.

In some embodiments of the disclosed uses, methods and kits, the IL-17 antagonist is an IL-17 antibody or antigen-binding fragment thereof. In some embodiments of the disclosed uses, methods and kits, the IL-17 antibody or antigen-binding fragment thereof is selected from the group consisting of: a) an IL-17 antibody or antigen-binding fragment thereof that binds to an epitope of human IL-17 comprising Leu74, Tyr85, His86, Met87, Asn88, Val124, Thr125, Pro126, Ile127, Val128, His129; b) an IL-17 antibody or antigen-binding fragment thereof that binds to an epitope of human IL-17 comprising Tyr43, Tyr44, Arg46, Ala79, Asp80; c) an IL-17 antibody or antigen-binding fragment thereof that binds to an epitope of an IL-17 homodimer having two mature human IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain; d) an IL-17 antibody or antigen-binding fragment thereof that binds to an epitope of an IL-17 homodimer having two mature human IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody or antigen-binding fragment thereof has a K_{D} of about 100-200 pM, and wherein the IL-17 antibody or antigen-binding fragment thereof has an in vivo half-life of about 23 to about 35 days; e) an IL-17 antibody that binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody has a K_{D} of about 100-200 pM as measured by a biosensor system (e.g., BIACORE®) or surface plasmon resonance, and wherein the IL-17 antibody has an in vivo half-life of about 23 to about 30 days; and f) an IL-17 antibody or antigen-binding fragment thereof comprising: i) an immunoglobulin heavy chain variable domain (V_{H}) comprising the amino acid sequence set forth as SEQ ID NO: 8; ii) an immunoglobulin light chain variable domain (V_{L}) comprising the amino acid sequence set forth as SEQ ID NO:10; iii) an immunoglobulin V_{H} domain comprising the amino acid sequence set forth as SEQ ID NO:8 and an immunoglobulin V_{L} domain comprising the amino acid sequence set forth as SEQ ID NO:10; iv) an immunoglobulin V_{H} domain comprising the hypervariable regions set forth as SEQ ID NO:1, SEQ ID NO:2, and SEQ ID NO:3; v) an immunoglobulin V_{L} domain comprising the hypervariable regions set forth as SEQ ID NO:4, SEQ ID NO:5 and SEQ ID NO:6; vi) an immunoglobulin V_{H} domain comprising the hypervariable regions set forth as SEQ ID NO:11, SEQ ID NO:12 and SEQ ID NO:13; vii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; viii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6; ix) an immunoglobulin light chain comprising the amino acid sequence set forth as SEQ ID NO:14; x) an immunoglobulin heavy chain comprising the amino acid sequence set forth as SEQ ID NO:15; or xi) an immunoglobulin light chain comprising the amino acid sequence set forth as SEQ ID NO:14 and an immunoglobulin heavy chain comprising the amino acid sequence set forth as SEQ ID NO:15.

In some embodiments of the disclosed uses, methods and kits, the IL-17 antibody or antigen-binding fragment thereof is a human or humanized antibody. In preferred embodiments of the disclosed uses, methods and kits, the IL-17 antibody or antigen-binding fragment thereof is secukinumab.

In preferred embodiments, the IL-17 antagonist (e.g., IL-17 antibody or antigen-binding fragment thereof, such as secukinumab) is subcutaneously (SC) administered at a dose of 150 mg or 300 mg. In other embodiments, the IL-17 antagonist (e.g., IL-17 antibody or antigen-binding fragment thereof, such as secukinumab) is intravenously (IV) administered at a dose of 6 mg/kg or 3 mg/kg.

In some embodiments, the IL-17 antagonist (e.g., IL-17 antibody or antigen-binding fragment thereof, such as secukinumab) is administered using an induction regimen, followed by a maintenance regimen. In some embodiments, the induction regimen comprises weekly administration and the maintenance regimen comprises administration every two weeks, every four weeks (monthly), or every eight weeks (every other month). In some embodiments, the induction regimen comprises a single administration and the maintenance regimen comprises administration every four weeks (monthly). In some embodiments, the induction regimen comprises every four weeks (monthly) administration and the maintenance regimen comprises administration every eight weeks (every other month).

In some embodiments, the IL-17 antagonist (e.g., IL-17 antibody or antigen-binding fragment thereof, such as secukinumab) is administered SC at a dose of about 300 mg during the induction and maintenance regimen. In some embodiments, the IL-17 antagonist (e.g., IL-17 antibody or antigen-binding fragment thereof, such as secukinumab) is administered SC at a dose of about 150 mg during the induction and maintenance regimen

In some embodiments, the IL-17 antagonist (e.g., IL-17 antibody or antigen-binding fragment thereof, such as secukinumab) is administered IV at a dose of about 6 mg/kg during the induction regimen. In some embodiments, the IL-17 antagonist (e.g., IL-17 antibody or antigen-binding fragment thereof, such as secukinumab) is administered IV at a dose of about 3 mg/kg during the maintenance regimen.

### BRIEF DESCRIPTION OF THE FIGURES

FIG. 1 provides the study design of a secukinumab-based human clinical trial for lupus nephritis.

### DETAILED DESCRIPTION OF THE DISCLOSURE

As used herein, IL-17 refers to interleukin-17A (IL-17A).

The term “comprising” encompasses “including” as well as “consisting,” e.g., a composition “comprising” X may consist exclusively of X or may include something additional, e.g., X+Y.

Unless otherwise specifically stated or clear from context, as used herein, the term “about” in relation to a numerical value is understood as being within the normal tolerance in the art, e.g., within two standard deviations of the mean. Thus, “about” can be within +/−10%, 9%, 8%, 7%, 6%, 5%, 4%, 3%, 2%, 1%, 0.1%, 0.05%, or 0.01% of the stated value, preferably +/−10% of the stated value. When used in front of a numerical range or list of numbers, the term “about” applies to each number in the series, e.g., the phrase “about 1-5” should be interpreted as “about 1—about 5”, or, e.g., the phrase “about 1, 2, 3, 4” should be interpreted as “about 1, about 2, about 3, about 4, etc.”

The word “substantially” does not exclude “completely,” e.g., a composition which is “substantially free” from Y may be completely free from Y. Where necessary, the word “substantially” may be omitted from the definition of the disclosure.

The term “antibody” as referred to herein includes naturally-occurring and whole antibodies. A naturally-occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed hypervariable regions or complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. Exemplary antibodies include secukinumab (Table 1), antibody XAB4 (U.S. Pat. No. 9,193,788), and ixekizumab (U.S. Pat. No. 7,838,638), the disclosures of which are incorporated by reference herein in their entirety.

The term “antigen-binding fragment” of an antibody, as used herein, refers to fragments of an antibody that retain the ability to specifically bind to an antigen (e.g., IL-17). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, CL and CH1 domains; a F(ab)2 fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated CDR. Exemplary antigen-binding fragments include the CDRs of secukinumab as set forth in SEQ ID NOs: 1-6 and 11-13 (Table 1), preferably the heavy chain CDR3. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies are also intended to be encompassed within the term “antibody”. Single chain antibodies and antigen-binding portions are obtained using conventional techniques known to those of skill in the art.

An “isolated antibody”, as used herein, refers to an antibody that is substantially free of other antibodies having different antigenic specificities (e.g., an isolated antibody that specifically binds IL-17 is substantially free of antibodies that specifically bind antigens other than IL-17). The term “monoclonal antibody” or “monoclonal antibody composition” as used herein refer to a preparation of antibody molecules of single molecular composition. The term “human antibody”, as used herein, is intended to include antibodies having variable regions in which both the framework and CDR regions are derived from sequences of human origin. A “human antibody” need not be produced by a human, human tissue or human cell. The human antibodies of the disclosure may include amino acid residues not encoded by human sequences (e.g., mutations introduced by random or site-specific mutagenesis in vitro, by N-nucleotide addition at junctions in vivo during recombination of antibody genes, or by somatic mutation in vivo). In some embodiments of the disclosed processes and compositions, the IL-17 antibody is a human antibody, an isolated antibody, and/or a monoclonal antibody.

The term “IL-17” refers to IL-17A, formerly known as CTLA8, and includes wild-type IL-17A from various species (e.g., human, mouse, and monkey), polymorphic variants of IL-17A, and functional equivalents of IL-17A. Functional equivalents of IL-17A according to the present disclosure preferably have at least about 65%, 75%, 85%, 95%, 96%, 97%, 98%, or even 99% overall sequence identity with a wild-type IL-17A (e.g., human IL-17A), and substantially retain the ability to induce IL-6 production by human dermal fibroblasts.

The term “K_{D}” is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods established in the art. A preferred method for determining the K_{D} of an antibody is by using surface plasmon resonance, or using a biosensor system, e.g., a BIACORE® system. In some embodiments, the IL-17 antibody or antigen-binding fragment thereof, e.g., secukinumab, binds human IL-17 with a K_{D} of about 100-250 pM.

The term “affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity. Standard assays to evaluate the binding affinity of the antibodies toward IL-17 of various species are known in the art, including for example, ELISAs, western blots and RIAs. The binding kinetics (e.g., binding affinity) of the antibodies also can be assessed by assays known in the art, e.g., using BIACORE® analysis or surface plasmon resonance.

An antibody that “inhibits” one or more of these IL-17 functional properties (e.g., biochemical, immunochemical, cellular, physiological or other biological activities, or the like) as determined according to methodologies known to the art and described herein, will be understood to relate to a statistically significant decrease in the particular activity relative to that seen in the absence of the antibody (or when a control antibody of irrelevant specificity is present). An antibody that inhibits IL-17 activity affects a statistically significant decrease, e.g., by at least about 10% of the measured parameter, by at least 50%, 80% or 90%, and in certain embodiments of the disclosed methods and compositions, the IL-17 antibody used may inhibit greater than 95%, 98% or 99% of IL-17 functional activity.

“Inhibit IL-6” as used herein refers to the ability of an IL-17 antibody or antigen-binding fragment thereof (e.g., secukinumab) to decrease IL-6 production from primary human dermal fibroblasts. The production of IL-6 in primary human (dermal) fibroblasts is dependent on IL-17 (Hwang et al., (2004) Arthritis Res Ther; 6:R120-128). In short, human dermal fibroblasts are stimulated with recombinant IL-17 in the presence of various concentrations of an IL-17 binding molecule or human IL-17 receptor with Fc part. The chimeric anti-CD25 antibody Simulect® (basiliximab) may be conveniently used as a negative control. Supernatant is taken after 16 h stimulation and assayed for IL-6 by ELISA. An IL-17 antibody or antigen-binding fragment thereof, e.g., secukinumab, typically has an IC₅₀ for inhibition of IL-6 production (in the presence 1 nM human IL-17) of about 50 nM or less (e.g., from about 0.01 to about 50 nM) when tested as above, i.e., said inhibitory activity being measured on IL-6 production induced by hu-IL-17 in human dermal fibroblasts. In some embodiments of the disclosed methods and compositions, IL-17 antibodies or antigen-binding fragments thereof, e.g., secukinumab, and functional derivatives thereof have an IC₅₀ for inhibition of IL-6 production as defined above of about 20 nM or less, more preferably of about 10 nM or less, more preferably of about 5 nM or less, more preferably of about 2 nM or less, more preferably of about 1 nM or less.

The term “derivative”, unless otherwise indicated, is used to define amino acid sequence variants, and covalent modifications (e.g., pegylation, deamidation, hydroxylation, phosphorylation, methylation, etc.) of an IL-17 antibody or antigen-binding fragment thereof, e.g., secukinumab, according to the present disclosure, e.g., of a specified sequence (e.g., a variable domain). A “functional derivative” includes a molecule having a qualitative biological activity in common with the disclosed IL-17 antibodies. A functional derivative includes fragments and peptide analogs of an IL-17 antibody as disclosed herein. Fragments comprise regions within the sequence of a polypeptide according to the present disclosure, e.g., of a specified sequence. Functional derivatives of the IL-17 antibodies disclosed herein (e.g., functional derivatives of secukinumab) preferably comprise V_{H} and/or V_{L} domains that have at least about 65%, 75%, 85%, 95%, 96%, 97%, 98%, or even 99% overall sequence identity with the V_{H} and/or V_{L} sequences of the IL-17 antibodies and antigen-binding fragments thereof disclosed herein (e.g., the V_{H} and/or V_{L} sequences of Table 1), and substantially retain the ability to bind human IL-17 or, e.g., inhibit IL-6 production of IL-17 induced human dermal fibroblasts.

The phrase “substantially identical” means that the relevant amino acid or nucleotide sequence (e.g., V_{H} or V_{L} domain) will be identical to or have insubstantial differences (e.g., through conserved amino acid substitutions) in comparison to a particular reference sequence. Insubstantial differences include minor amino acid changes, such as 1 or 2 substitutions in a 5 amino acid sequence of a specified region (e.g., V_{H} or V_{L} domain). In the case of antibodies, the second antibody has the same specificity and has at least 50% of the affinity of the same. Sequences substantially identical (e.g., at least about 85% sequence identity) to the sequences disclosed herein are also part of this application. In some embodiments, the sequence identity of a derivative IL-17 antibody (e.g., a derivative of secukinumab, e.g., a secukinumab biosimilar antibody) can be about 90% or greater, e.g., 90%, 91%, 92%, 93%, 94%, 95%, 96%, 97%, 98%, 99% or higher relative to the disclosed sequences.

“Identity” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent identity, and not considering any conservative substitutions as part of the sequence identity. Neither N- or C-terminal extensions nor insertions shall be construed as reducing identity. Methods and computer programs for the alignment are known. The percent identity can be determined by standard alignment algorithms, for example, the Basic Local Alignment Search Tool (BLAST) described by Altshul et al. ((1990) J. Mol. Biol., 215: 403 410); the algorithm of Needleman et al. ((1970) J. Mol. Biol., 48: 444 453); or the algorithm of Meyers et al. ((1988) Comput. Appl. Biosci., 4: 11 17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

“Amino acid(s)” refer to all naturally occurring L-α-amino acids, e.g., and include D-amino acids. The phrase “amino acid sequence variant” refers to molecules with some differences in their amino acid sequences as compared to the sequences according to the present disclosure. Amino acid sequence variants of an antibody according to the present disclosure, e.g., of a specified sequence, still have the ability to bind the human IL-17 or, e.g., inhibit IL-6 production of IL-17 induced human dermal fibroblasts. Amino acid sequence variants include substitutional variants (those that have at least one amino acid residue removed and a different amino acid inserted in its place at the same position in a polypeptide according to the present disclosure), insertional variants (those with one or more amino acids inserted immediately adjacent to an amino acid at a particular position in a polypeptide according to the present disclosure) and deletional variants (those with one or more amino acids removed in a polypeptide according to the present disclosure).

The term “pharmaceutically acceptable” means a nontoxic material that does not interfere with the effectiveness of the biological activity of the active ingredient(s).

The term “administering” in relation to a compound, e.g., an IL-17 binding molecule or another agent, is used to refer to delivery of that compound to a patient by any route.

As used herein, a “therapeutically effective amount” refers to an amount of an IL-17 antagonist, e.g., IL-17 binding molecule (e.g., IL-17 antibody or antigen-binding fragment thereof, e.g., secukinumab) or IL-17 receptor binding molecule (e.g., IL-17 antibody or antigen-binding fragment thereof) that is effective, upon single or multiple dose administration to a patient (such as a human) for treating, preventing, preventing the onset of, curing, delaying, reducing the severity of, ameliorating at least one symptom of a disorder or recurring disorder, or prolonging the survival of the patient beyond that expected in the absence of such treatment. When applied to an individual active ingredient (e.g., an IL-17 antagonist, e.g., secukinumab) administered alone, the term refers to that ingredient alone. When applied to a combination, the term refers to combined amounts of the active ingredients that result in the therapeutic effect, whether administered in combination, serially or simultaneously.

The term “treatment” or “treat” is herein defined as the application or administration of an IL-17 antibody according to the disclosure, for example, secukinumab or ixekizumab, or a pharmaceutical composition comprising said anti-IL-17 antibody, to a subject or to an isolated tissue or cell line from a subject, where the subject has a particular disease (e.g., LN), a symptom associated with the disease (e.g., LN), or a predisposition towards development of the disease (e.g., LN) (if applicable), where the purpose is to cure (if applicable), delay the onset of, reduce the severity of, alleviate, ameliorate one or more symptoms of the disease, improve the disease, reduce or improve any associated symptoms of the disease or the predisposition toward the development of the disease. The term “treatment” or “treat” includes treating a patient suspected to have the disease as well as patients who are ill or who have been diagnosed as suffering from the disease or medical condition, and includes suppression of clinical relapse.

As used herein, the phrase “population of patients” is used to mean a group of patients. In some embodiments of the disclosed methods, the IL-17 antagonist (e.g., IL-17 antibody, such as secukinumab) is used to treat a population of LN patients.

As used herein, “selecting” and “selected” in reference to a patient is used to mean that a particular patient is specifically chosen from a larger group of patients on the basis of (due to) the particular patient having a predetermined criteria. Similarly, “selectively treating” refers to providing treatment to a patient having a particular disease, where that patient is specifically chosen from a larger group of patients on the basis of the particular patient having a predetermined criterion. Similarly, “selectively administering” refers to administering a drug to a patient that is specifically chosen from a larger group of patients on the basis of (due to) the particular patient having a predetermined criterion. By selecting, selectively treating and selectively administering, it is meant that a patient is delivered a personalized therapy based on the patient's personal history (e.g., prior therapeutic interventions, e.g., prior treatment with biologics), biology (e.g., particular genetic markers), and/or manifestation (e.g., not fulfilling particular diagnostic criteria), rather than being delivered a standard treatment regimen based solely on the patient's membership in a larger group. Selecting, in reference to a method of treatment as used herein, does not refer to fortuitous treatment of a patient having a particular criterion, but rather refers to the deliberate choice to administer treatment to a patient based on the patient having a particular criterion. Thus, selective treatment/administration differs from standard treatment/administration, which delivers a particular drug to all patients having a particular disease, regardless of their personal history, manifestations of disease, and/or biology. In some embodiments, the patient is selected for treatment based on having LN, e.g., ISN/RPS Class III or IV LN. In some embodiments, the patient is selected for treatment based on having active LN. In some embodiments, the patient is selected for treatment based on having previously had an inadequate response to a standard-of-care LN therapy.

### IL-17 Antagonists

The various disclosed processes, kits, uses and methods utilize an IL-17 antagonist, e.g., IL-17 binding molecule (e.g., soluble IL-17 receptor, IL-17 antibody or antigen-binding fragment thereof, e.g., secukinumab) or IL-17 receptor binding molecule (e.g., IL-17 receptor antibody or antigen-binding fragment thereof). In some embodiments, the IL-17 antagonist is an IL-17 binding molecule, preferably an IL-17 antibody or antigen-binding fragment thereof.

In one embodiment, the IL-17 antibody or antigen-binding fragment thereof comprises at least one immunoglobulin heavy chain variable domain (V_{H}) comprising hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3. In one embodiment, the IL-17 antibody or antigen-binding fragment thereof comprises at least one immunoglobulin light chain variable domain (V_{L}′) comprising hypervariable regions CDR1′, CDR2′ and CDR3′, said CDR1′ having the amino acid sequence SEQ ID NO:4, said CDR2′ having the amino acid sequence SEQ ID NO:5 and said CDR3′ having the amino acid sequence SEQ ID NO:6. In one embodiment, the IL-17 antibody or antigen-binding fragment thereof comprises at least one immunoglobulin heavy chain variable domain (V_{H}) comprising hypervariable regions CDR1-x, CDR2-x and CDR3-x, said CDR1-x having the amino acid sequence SEQ ID NO:11, said CDR2-x having the amino acid sequence SEQ ID NO:12, and said CDR3-x having the amino acid sequence SEQ ID NO:13.

In one embodiment, the IL-17 antibody or antigen-binding fragment thereof comprises at least one immunoglobulin V_{H} domain and at least one immunoglobulin V_{L} domain, wherein: a) the immunoglobulin V_{H} domain comprises (e.g., in sequence): i) hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3; or ii) hypervariable regions CDR1-x, CDR2-x and CDR3-x, said CDR1-x having the amino acid sequence SEQ ID NO:11, said CDR2-x having the amino acid sequence SEQ ID NO:12, and said CDR3-x having the amino acid sequence SEQ ID NO:13; and b) the immunoglobulin V_{L} domain comprises (e.g., in sequence) hypervariable regions CDR1′, CDR2′ and CDR3′, said CDR1′ having the amino acid sequence SEQ ID NO:4, said CDR2′ having the amino acid sequence SEQ ID NO:5, and said CDR3′ having the amino acid sequence SEQ ID NO:6.

In one embodiment, the IL-17 antibody or antigen-binding fragment thereof comprises: a) an immunoglobulin heavy chain variable domain (V_{H}) comprising the amino acid sequence set forth as SEQ ID NO:8; b) an immunoglobulin light chain variable domain (V_{L}) comprising the amino acid sequence set forth as SEQ ID NO:10; c) an immunoglobulin V_{H} domain comprising the amino acid sequence set forth as SEQ ID NO: 8 and an immunoglobulin V_{L} domain comprising the amino acid sequence set forth as SEQ ID NO:10; d) an immunoglobulin V_{H} domain comprising the hypervariable regions set forth as SEQ ID NO:1, SEQ ID NO:2, and SEQ ID NO:3; e) an immunoglobulin V_{L} domain comprising the hypervariable regions set forth as SEQ ID NO:4, SEQ ID NO:5 and SEQ ID NO:6; f) an immunoglobulin V_{H} domain comprising the hypervariable regions set forth as SEQ ID NO:11, SEQ ID NO:12 and SEQ ID NO:13; g) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or h) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6.

For ease of reference the amino acid sequences of the hypervariable regions of the secukinumab monoclonal antibody, based on the Kabat definition and as determined by the X-ray analysis and using the approach of Chothia and coworkers, is provided in Table 1, below.

TABLE 1
Amino acid sequences of the hypervariable regions of secukinumab.
Light-Chain

| | | |
|---|---|---|
| CDR1′ | Kabat | R-A-S-Q-S-V-S-S-S-Y-L-A (SEQ ID NO: 4) |
| | Chothia | R-A-S-Q-S-V-S-S-S-Y-L-A (SEQ ID NO: 4) |
| CDR2′ | Kabat | G-A-S-S-R-A-T (SEQ ID NO: 5) |
| | Chothia | G-A-S-S-R-A-T (SEQ ID NO: 5) |
| CDR3′ | Kabat | Q-Q-Y-G-S-S-P-C-T (SEQ ID NO: 6) |
| | Chothia | Q-Q-Y-G-S-S-P-C-T (SEQ ID NO: 6) |

Heavy-Chain

| | | |
|---|---|---|
| CDR1 | Kabat | N-Y-W-M-N (SEQ ID NO: 1) |
| CDR1-x | Chothia | G-F-T-F-S-N-Y-W-M-N (SEQ ID NO: 11) |
| CDR2 | Kabat | A-I-N-Q-D-G-S-E-K-Y-Y-V-G-S-V-K-G (SEQ ID NO: 2) |
| CDR2-X | Chothia | A-I-N-Q-D-G-S-E-K-Y-Y (SEQ ID NO: 12) |
| CDR3 | Kabat | D-Y-Y-D-I-L-T-D-Y-Y-I-H-Y-W-Y-F-D-L (SEQ ID |
| | | NO: 3) |
| CDR3-X | Chothia | C-V-R-D-Y-Y-D-I-L-T-D-Y-Y-I-H-Y-W-Y-F-D-L-W-G |
| | | (SEQ ID NO: 13) |

Secukinumab CDRs according to IMGT are as follows: light chain CDR1 (QSVSSSY; SEQ ID NO:16), CDR 2 (GAS; SEQ ID NO:17), CDR3 (QQYGSSPCT; SEQ ID NO:18); and heavy chain CDR1 (GFTFSNYW; SEQ ID NO:19), CDR2 (INQDGSEK; SEQ ID NO:20), (VRDYYDILTDYYIHYWYFDL; SEQ ID NO:21).

In preferred embodiments, constant region domains also comprise suitable human constant region domains, for instance as described in “Sequences of Proteins of Immunological Interest”, Kabat E. A. et al, US Department of Health and Human Services, Public Health Service, National Institute of Health. The DNA encoding the V_{L} of secukinumab is set forth in SEQ ID NO:9. The DNA encoding the V_{H} of secukinumab is set forth in SEQ ID NO:7.

In some embodiments, the IL-17 antibody or antigen-binding fragment thereof (e.g., secukinumab) comprises the three CDRs of SEQ ID NO:10. In other embodiments, the IL-17 antibody or antigen-binding fragment thereof comprises the three CDRs of SEQ ID NO: 8. In other embodiments, the IL-17 antibody or antigen-binding fragment thereof comprises the three CDRs of SEQ ID NO:10 and the three CDRs of SEQ ID NO:8. CDRs according to Kabat and Chothia of SEQ ID NO:8 and SEQ ID NO:10 may be found in Table 1. CDRs according to IMGT are set forth as SEQ ID NOs:16-18 (light chain CDR1, CDR2, CDR3, respectively) and SEQ ID NOs:19-21 (light chain CDR1, CDR2, CDR3, respectively). The free cysteine in the light chain (CysL97) may be seen, e.g., in SEQ ID NO:6.

In some embodiments, IL-17 antibody or antigen-binding fragment thereof comprises the light chain of SEQ ID NO:14. In other embodiments, the IL-17 antibody or antigen-binding fragment thereof comprises the heavy chain of SEQ ID NO:15. In other embodiments, the IL-17 antibody or antigen-binding fragment thereof comprises the light chain of SEQ ID NO:14 and the heavy domain of SEQ ID NO:15. In some embodiments, the IL-17 antibody or antigen-binding fragment thereof comprises the three CDRs of SEQ ID NO:14. In other embodiments, IL-17 antibody or antigen-binding fragment thereof comprises the three CDRs of SEQ ID NO:15. In other embodiments, the IL-17 antibody or antigen-binding fragment thereof comprises the three CDRs of SEQ ID NO:14 and the three CDRs of SEQ ID NO:15. CDRs of SEQ ID NO:14 and SEQ ID NO:15 may be found in Table 1.

Hypervariable regions may be associated with any kind of framework regions, though preferably are of human origin. Suitable framework regions are described in Kabat E. A. et al, ibid. The preferred heavy chain framework is a human heavy chain framework, for instance that of the secukinumab antibody. It consists in sequence, e.g. of FR1 (amino acid 1 to 30 of SEQ ID NO:8), FR2 (amino acid 36 to 49 of SEQ ID NO:8), FR3 (amino acid 67 to 98 of SEQ ID NO:8) and FR4 (amino acid 117 to 127 of SEQ ID NO:8) regions. Taking into consideration the determined hypervariable regions of secukinumab by X-ray analysis, another preferred heavy chain framework consists in sequence of FR1-x (amino acid 1 to 25 of SEQ ID NO:8), FR2-x (amino acid 36 to 49 of SEQ ID NO:8), FR3-x (amino acid 61 to 95 of SEQ ID NO:8) and FR4 (amino acid 119 to 127 of SEQ ID NO: 8) regions. In a similar manner, the light chain framework consists, in sequence, of FR1′ (amino acid 1 to 23 of SEQ ID NO:10), FR2′ (amino acid 36 to 50 of SEQ ID NO:10), FR3′ (amino acid 58 to 89 of SEQ ID NO:10) and FR4′ (amino acid 99 to 109 of SEQ ID NO:10) regions.

In one embodiment, the IL-17 antibody or antigen-binding fragment thereof (e.g., secukinumab) is selected from a human IL-17 antibody that comprises at least: a) an immunoglobulin heavy chain or fragment thereof which comprises a variable domain comprising, in sequence, the hypervariable regions CDR1, CDR2 and CDR3 and the constant part or fragment thereof of a human heavy chain; said CDR1 having the amino acid sequence SEQ ID NO:1, said CDR2 having the amino acid sequence SEQ ID NO:2, and said CDR3 having the amino acid sequence SEQ ID NO:3; and b) an immunoglobulin light chain or fragment thereof which comprises a variable domain comprising, in sequence, the hypervariable regions CDR1 CDR2′, and CDR3′ and the constant part or fragment thereof of a human light chain, said CDR1′ having the amino acid sequence SEQ ID NO:4, said CDR2′ having the amino acid sequence SEQ ID NO:5, and said CDR3′ having the amino acid sequence SEQ ID NO:6.

In one embodiment, the IL-17 antibody or antigen-binding fragment thereof is selected from a single chain antibody or antigen-binding fragment thereof that comprises an antigen-binding site comprising: a) a first domain comprising, in sequence, the hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3; and b) a second domain comprising, in sequence, the hypervariable regions CDR1′, CDR2′ and CDR3′, said CDR1′ having the amino acid sequence SEQ ID NO:4, said CDR2′ having the amino acid sequence SEQ ID NO:5, and said CDR3′ having the amino acid sequence SEQ ID NO:6; and c) a peptide linker which is bound either to the N-terminal extremity of the first domain and to the C-terminal extremity of the second domain or to the C-terminal extremity of the first domain and to the N-terminal extremity of the second domain.

Alternatively, an IL-17 antibody or antigen-binding fragment thereof as used in the disclosed methods may comprise a derivative of the IL-17 antibodies set forth herein by sequence (e.g., pegylated variants, glycosylation variants, affinity-maturation variants, etc.). Alternatively, the V_{H} or V_{L} domain of an IL-17 antibody or antigen-binding fragment thereof used in the disclosed methods may have V_{H} or V_{L} domains that are substantially identical to the V_{H} or V_{L} domains set forth herein (e.g., those set forth in SEQ ID NO:8 and 10). A human IL-17 antibody disclosed herein may comprise a heavy chain that is substantially identical to that set forth as SEQ ID NO:15 and/or a light chain that is substantially identical to that set forth as SEQ ID NO:14. A human IL-17 antibody disclosed herein may comprise a heavy chain that comprises SEQ ID NO:15 and a light chain that comprises SEQ ID NO:14. A human IL-17 antibody disclosed herein may comprise: a) one heavy chain which comprises a variable domain having an amino acid sequence substantially identical to that shown in SEQ ID NO:8 and the constant part of a human heavy chain; and b) one light chain which comprises a variable domain having an amino acid sequence substantially identical to that shown in SEQ ID NO:10 and the constant part of a human light chain.

Alternatively, an IL-17 antibody or antigen-binding fragment thereof used in the disclosed methods may be an amino acid sequence variant of the reference IL-17 antibodies set forth herein, as long as it contains CysL97. The disclosure also includes IL-17 antibodies or antigen-binding fragments thereof (e.g., secukinumab) in which one or more of the amino acid residues of the V_{H} or V_{L} domain of secukinumab (but not CysL97), typically only a few (e.g., 1-10), are changed; for instance by mutation, e.g., site directed mutagenesis of the corresponding DNA sequences. In all such cases of derivative and variants, the IL-17 antibody or antigen-binding fragment thereof is capable of inhibiting the activity of about 1 nM (=30 ng/ml) human IL-17 at a concentration of about 50 nM or less, about 20 nM or less, about 10 nM or less, about 5 nM or less, about 2 nM or less, or more preferably of about 1 nM or less of said molecule by 50%, said inhibitory activity being measured on IL-6 production induced by hu-IL-17 in human dermal fibroblasts as described in Example 1 of WO 2006/013107.

In some embodiments, the IL-17 antibodies or antigen-binding fragments thereof, e.g., secukinumab, bind to an epitope of mature human IL-17 comprising Leu74, Tyr85, His86, Met87, Asn88, Val124, Thr125, Pro126, Ile127, Val128, His129. In some embodiments, the IL-17 antibody, e.g., secukinumab, binds to an epitope of mature human IL-17 comprising Tyr43, Tyr44, Arg46, Ala79, Asp80. In some embodiments, the IL-17 antibody, e.g., secukinumab, binds to an epitope of an IL-17 homodimer having two mature human IL-17 chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain. The residue numbering scheme used to define these epitopes is based on residue one being the first amino acid of the mature protein (i.e., IL-17A lacking the 23 amino acid N-terminal signal peptide and beginning with glycine). The sequence for immature IL-17A is set forth in the Swiss-Prot entry Q16552. In some embodiments, the IL-17 antibody has a K_{D} of about 100-200 pM (e.g., as determined by a BIACORE® assay or surface plasmon resonance). In some embodiments, the IL-17 antibody has an IC₅₀ of about 0.4 nM for in vitro neutralization of the biological activity of about 0.67 nM human IL-17A. In some embodiments, the absolute bioavailability of subcutaneously (SC) administered IL-17 antibody has a range of about 60%—about 80%, e.g., about 76%. In some embodiments, the IL-17 antibody, such as secukinumab, has an elimination half-life of about 4 weeks (e.g., about 23 to about 35 days, about 23 to about 30 days, e.g., about 30 days). In some embodiments, the IL-17 antibody (such as secukinumab) has a Tₘₐₓ of about 7-8 days.

Particularly preferred IL-17 antibodies or antigen-binding fragments thereof used in the disclosed methods are human antibodies, especially secukinumab as described in Examples 1 and 2 of WO 2006/013107. Other preferred IL-17 antibodies for use in the disclosed methods, kits and regimens are those set forth in U.S. Pat. Nos. 8,057,794; 8,003,099; 8,110,191; and 7,838,638 and US Published Patent Application Nos: 20120034656 and 20110027290, which are incorporated by reference herein in their entirety.

### Methods of Treatment and Uses of IL-17 Antagonists

The disclosed IL-17 antagonists, e.g., IL-17 binding molecules (e.g., IL-17 antibody or antigen-binding fragment thereof, e.g., secukinumab) or IL-17 receptor binding molecules (e.g., IL-17 receptor antibody or antigen-binding fragment thereof), may be used in vitro, ex vivo, or incorporated into pharmaceutical compositions and administered in vivo to treat LN patients (e.g., human patients).

LN is categorized histologically into six classes by the International Society of Nephrology/Renal Pathology Society (ISN/RPS) classification system that has become the standard for renal biopsy interpretation because of improved correlation with prognostic and therapeutic outcomes (Weening et al., 2004; J Am Soc Nephrol; 15(2):241-50; Markowitz et al., 2007 Kidney Int; 71(6):491). Treatments include management with corticosteroids for lower stage disease, followed by more aggressive immunosuppressive therapies for more severe disease and ultimately renal transplant.

Class I and II LN is present in approximately 10.2 to 25.7% of patients with LN and is characterized by immune-complexes that form within the mesangium by binding of antibodies to autoantigens (Wang et al., 2018 Arch Rheumatol; 33(1):17-25). Patients with class I minimal mesangial LN display normal glomeruli by light microscopy, but mesangial immune deposits are visible by immunofluorescence. Patients with LN class I and II usually have a more favorable prognosis than with other classes of LN. Class I and II LN are usually managed with corticosteroids (Yu et al., 2017 Nat Rev Nephrol; 13(8):483-495).

Class III and IV LN is detected in approximately 39 to 71.9% of LN patients and is the result of the deposition of immune complexes in the subendothelial space of the glomerular capillaries (Wang et al., 2018 Arch Rheumatol; 33(1):17-25). Both classes are considered to have similar lesions that differ by severity and distribution. Class IV diffuse LN is distinguished from class III on the basis of involvement of more than 50% of glomeruli with endocapillary lesions. Patients with class III and IV LN require aggressive therapy with glucocorticoids and immunosuppressive agents (Hahn et al. (2012) Arthritis Care Res 64:797-808).

Class V LN, also known as membranous lupus nephritis, is present in approximately 12.1 to 20.3% of patients with LN and is characterized by the deposition of immune complexes in the subepithelial compartment of the glomeruli (Wang et al., 2018 Arch Rheumatol; 33(1):17-25). Class V LN, when combined with III or IV, should be treated in the same manner as III or IV.

Class VI LN represents 1.3 to 4.7% of LN patients and is characterized by the development of sclerotic lesions and leads to irreversible glomerulosclerosis (Wang et al., 2018 Arch Rheumatol; 33(1):17-25). With class VI LN, the progression of renal fibrosis and sclerosis is usually associated with a progressive decline in glomerular filtration rate and ultimately the development of ESRD. Histologic class VI (sclerosis of ≥90% of glomeruli) generally requires preparation for renal replacement therapy rather than immunosuppression.

Class III and IV LN have subgroups of “A” (active lesions), “C” (chronic lesions) and “A/C” (active and chronic lesions). (Hahn et al. (2012)). As per the revision of the pathological classification of LN, categorizing class IV into segmental or global subdivisions (“IV-S” and “IV-G”) are to be eliminated due to limitation of reproducibility of the information and weak clinical significance. The newly proposed modifications of the NIH LN activity and chronicity scoring system also recommends a semi-quantitative approach to describe active and chronic lesions instead of “A”, “C”, and “A/C” parameters and new definitions for mesangial hypercellularity and for cellular, fibrocellular, and fibrous crescents (Bajema et al (2018). Kidney International; 93(4):789-796).

In some embodiments, the LN patient to be treated using the disclosed methods, uses, kits, etc. has International Society of Nephrology/Renal Pathology Society (ISN/RPS) Class III or IV LN. In some embodiments, the LN patient to be treated using the disclosed methods, uses, kits, etc. has ISN/RPS Class III or IV LN with or without co-existing features of Class V LN. In some embodiments, the LN patient to be treated using the disclosed methods, uses, kits, etc. has ISN/RPS Class III or IV LN, but not Class III(C), Class IV-S(C) or IV-G(C) LN. In other embodiments, the LN patient to be treated using the disclosed methods, uses, kits, etc. has ISN/RPS Class III or IV LN, but not chronic Class III or Class IV LN. As used herein, the phrase “features of Class V LN” refers to the disease aspects (e.g., histological, pathological, etc.) of Class V LN as provided by the ISN/RPS (see, e.g., Weening et a. (2004) Kidney Int. 65:521-530 and Weening et a. (2004) J Am Soc Nephrol. 15:241-250).

In some embodiments of the disclosed methods, kits, and uses, the LN patient to be treated has a renal biopsy showing active glomerulonephritis WHO or ISN/RPS Class III or IV LN [excluding III (C), IV-S(C) and IV-G (C)], with or without co-existing class V features, and whose disease has been inadequately controlled with previous SoC treatment(s).

As used herein, the phrase “active LN” refers to LN of the following criteria: biopsy results indicating active glomerulonephritis WHO or ISN/RPS Class III or IV LN [excluding III (C), IV-S(C) and IV-G (C)], with or without co-existing Class V; UPCR≥1 prior to treatment; estimated eGFR>30 mL/min/1.73 m2 by Chronic Kidney Disease Epidemiology Collaboration (CKD-EPI) (see Levy et al. (2009) Ann Intern Med 150(9):604-612; Martinez-Martinez (2012) Rheumatol Int 32: 2293); and active urinary sediment (presence of cellular casts (granular or red blood cell casts) or hematuria (>5 red blood cells per high power field)). In some embodiments of the disclosed methods, kits, and uses, the LN patient to be treated has active LN.

As used herein, the phrases “inadequately controlled”, “inadequate response”, and the like refer to treatments that produce an insufficient response in a patient, e.g., the LN patient still has one or more pathological symptoms of LN, e.g., renal dysfunction, nephrotic syndrome, elevated urinary cast, urine protein, elevated urinary sediment, hematuria, nephropathy, etc. In some embodiments, prior to administering the IL-17 antagonist, the patient has had an inadequate response to prior treatment with a standard-of-care LN therapy. In some embodiments of the disclosure, an inadequate response is indicated by the LN patient having a UPCR≥1 and active urinary sediment (presence of cellular [granular or red blood cell] cast) or hematuria (>5 red blood cells per high power field). In some embodiments, the LN patient to be treated using the disclosed methods, uses, kits, etc. has LN that has been inadequately controlled with previous SoC treatment(s).

A patient who has responded adequately to treatment with a standard-of-care LN therapy but has discontinued due to a side effect is termed “intolerant”. In some embodiments, the LN patient to be treated using the disclosed methods, uses, kits, etc. is intolerant to a standard-of-care LN therapy.

As used herein, “standard-of-care LN therapy” refers to a treatment regimen employing LN agents typically employed by health care professionals, including immunosuppressants and steroids (e.g., corticosteroids, e.g., glucocorticoids, e.g., prednisolone, prednisone, methylprednisolone, etc.), e.g., mycophenolate mofetil (MMF), cyclosporine A, rituximab, ocrelizumab, abatacept, azathioprine, calcineurin inhibitors, cyclosporine A, tacrolimus, cyclophosphamide (CYC), mycophenolic acid (MPA) (including salts thereof), voclosporin, belimumab, ustekinumab, iguratimod, anifrolumab, BI655064, CFZ533, and combination thereof. Steroids for treating LN may be given by IV pulse or orally, and are preferably corticosteroids, e.g., glucocorticoids, e.g., prednisolone, prednisone, methylprednisolone, etc. Doses and regimens of these LN agents (both induction and maintenance doses and regimens) are known to clinicians and may be found in, e.g., Hahn et al. (2012) Arthritis Care Res (Hoboken) 64(6): 797-808. In some embodiments, LN steroid therapy comprises pulse intravenous corticosteroid therapy where indicated, e.g., 500-1000 mg methylprednisolone daily for three doses, followed by daily oral glucocorticoids (0.5-1 mg/kg/day). In some embodiments, LN immunosuppressant therapy comprises an MMF dose of up to 3 g daily. In some embodiments, LN immunosuppressant therapy comprises a CYC dose of up to 15 mg/kg daily. As used herein, “mycophenolic acid (MPA)” refers to mycophenolate mofetil (MMF) or enteric-coated MPA sodium at equivalent dose. In some embodiments, during treatment with the IL-17 antibody or antigen-binding fragment, the dose of MPA administered to the patient is reduced, and the patient does not experience a flare as a result of said reduction.

The most preferred standard-of-care LN therapy employs MPA (MMF or enteric coated MPA sodium) or CYC, along with corticosteroids for class III/IV LN patients for induction (Hahn et al (2012) Arthritis Care Res 64:797-808; Bertsias et al (2012) Ann Rheum Dis; 71, 1771-1782) as well as maintenance therapy after inducing remission (Palmer et al (2017) Am J Kidney Dis; 70(3):324-336). For example:
low-dose CYC induction treatment typically consists of 6 administrations of 500 mg intravenous (i.v.) CYC every 2 weeks;
MMF induction dose is typically up to 3 g daily (preferably 2 g daily) or equivalent dosage of enteric coated MPA sodium up to 2,160 mg daily (preferably 1440 mg daily) (Zeher et al (2011) Lupus 20(14):1484-93; Jones et al (2014) Clin Kidney J (2014) 7: 562-568) is favored for those patients with class III/IV and crescents, and for those patients with proteinuria and a recent significant rise in creatinine.
Pulse i.v. corticosteroid is typically 500-1000 mg methylprednisolone daily for 3 doses, followed by daily oral glucocorticoids (0.3-1 mg/kg/day, preferably 0.3 mg/kg/day— 0.5 mg/kg/day) followed by a taper to the minimal amount necessary to control disease.

As used herein, “induction” refers to the portion of a LN therapy that induces remission of the disease. Preferred induction treatments include administration of MPA or CYC to the patient. Induction for MPA is typically 6 months and for CYC is typically 12 weeks. Thereafter, a patient is treated with a “maintenance” regimen to maintain the patient in a disease-free (or relapse-free) state. A typical standard-of-care LN therapy may employ, e.g., induction: MMF 2-3 g per day for 6 months or CYC+glucocorticoid IV pulse for 3 days, then prednisone orally at 0.5-1 mg/kg per day tapered after a few weeks to the lowest effective dose; maintenance (if improvement after induction): MMF 1-2 g per day or AZA 2 mg/kg/day+−low-dose daily glucocorticoid. In some embodiments, the target dose during the maintenance period is 1-2 g/day of MMF or of equivalent dosage of enteric-coated MPA. Further reduction of MMF to 0.5 g/day or of equivalent dosage of enteric-coated MPA is also within the scope of the disclosure. In some embodiments, patients will also receive a maintenance dose of oral corticosteroids, with a target dose of 5 mg/day (2.5-7.5 mg/day acceptable dose range) from Week 16.

In one embodiments of the disclosure, the IL-17 antibody or antigen-binding fragment (e.g., secukinumab) is employed during maintenance therapy as an “add-on” to standard-of-care in adult patients with active LN. In other embodiments of the disclosure, the IL-17 antibody or antigen-binding fragment (e.g., secukinumab) is employed during both induction and maintenance therapy as an “add-on” to standard-of-care in adult patients with active LN.

As used herein the term “flare,” in the context of a LN flare (also referred to as a “renal flare”) is as described in Parikh et al. (2014) Clin. J. Am. Soc. Nephrol. 9(2):279-84, i.e., an increase in LN disease activity requiring alternative or more intensive treatment. In some embodiments of the disclosure, treatment according to the disclosed methods, kits, uses, etc. with the IL-17 antagonist (e.g., secukinumab) prevents LN flares, decreases the severity of LN flares, and/or decreases the frequency of LN flares.

The effectiveness of an LN treatment may be assessed using various known methods and tools that measure kidney disease state and/or kidney activity. Such tests include, e.g., glomerular filtration rate (GFR) or estimated GFR (eGFR), serum creatinine measurements, measurement of cellular casts, determination of urinary protein: urinary creatinine ratio (UPCR).

A urinary protein: urinary creatinine ratio (UPCR) (preferably done as part of a 24-hour urine test) refers to a diagnostic test that examines the ratio of the level of protein to creatinine in a sample from a patient's urine.

An estimated glomerular filtration rate (eGFR) may be measured by the Chronic Kidney Disease Epidemiology Collaboration (CKD-EPI) equation (Martinez-Martinez et al. (2012) Nefrologia 33(1):99-106); Levey et al. (2009) Ann Intern Med. 150(9) 604-12))

In some embodiments, the LN patient achieves a complete renal response (CRR) or a partial renal response (PRR).

As used herein, the phrase “complete renal response (CRR)” refers to a preferred outcome for therapy in LN, e.g., using the disclosed IL-17 antagonists (e.g., secukinumab). It is demonstrated by clinically significant improvement of renal function. In preferred embodiments, CRR is achieved when the following two conditions are met: 1) estimated glomerular filtration rate (eGFR) is within the normal range or no less than 85% of baseline; and 2) 24-hour urinary protein to creatinine ratio (UPCR)≤0.5 mg/mg.

By “adequate response to a steroid daily dose” is meant that the patient does not experience a relapse or LN flare wile treated with a particular daily dose of steroid. The dose that achieves this adequate response is referred to as a “stable dose”. As used herein, the phrase “achieve a daily steroid dose of X following a steroid tapering regimen” means that a patient can utilize a stable steroid dose X after an original dose is tapered to X.

As used herein “steroid tapering”, “taper”, “tapering regimen” and the like refer to a reduction regimen of a steroid (e.g., corticosteroid, e.g., glucocorticoid, e.g., prednisone, prednisolone, methylprednisolone) given to a patient over time. The tapering schedule (timing and dose decrease) will depend on the original steroid (e.g., corticosteroid, e.g., glucocorticoid, e.g., prednisone, prednisolone, methylprednisolone) dose the patient is taking prior to treatment with the IL-17 antibody or antigen-binding fragment. A tapering regimen is in alignment with common medical practice in LN and is designed to minimize steroid related toxicity. Steroid tapering is a key goal to achieve in patients with LN given that the current SoC LN treatment regimens have substantial side effects from glucocorticoids and prolonged immunosuppression (Schwartz (2014). Curr Opin Rheumatol; 26: 502-509). In some embodiments of the disclosure, during treatment with the IL-17 antibody or antigen-binding fragment, the dose of steroid (e.g., corticosteroid, e.g., glucocorticoid, e.g., prednisone, prednisolone, methylprednisolone) administered to the patient is reduced using a taper regimen, and the patient does not experience a flare as a result of said reduction. In some embodiments of the disclosure, when said method is used to treat a population of patients with LN, at least 50% of said patients achieve a daily steroid dose of ≤10 mg/day following a steroid tapering regimen during treatment with the IL-17 antibody or antigen-binding fragment. In some embodiments of the disclosure, when said method is used to treat a population of patients with LN, at least 50% of said patients achieve a daily steroid dose of ≤5 mg/day following a steroid tapering regimen during treatment with the IL-17 antibody or antigen-binding fragment.

As used herein, the phrase “partial renal response (PRR)” refers to a preferred outcome for therapy in LN. PRR, adapted from Bertsias et al (2012) Ann Rheum Dis; 71, 1771-1782, is defined as: 1. ≥50% reduction in proteinuria to sub-nephrotic levels; and 2. normal or near-normal eGFR (≥85% of baseline) is achieved no later than 12 months following treatment initiation. PRR, adapted from Wofsy et al. (2013) Arthritis Rheum; 65(6): 1586-1591, is defined as: 1. for patients with UPCR>3 at baseline, reduction in UPCR to <3; or for patients with UPCR≤3 at baseline, reduction in UPCR of at least 50% or final UPCR<1; and 2. reduced serum creatinine relative to baseline or an increase in serum creatinine of not more than 15% above baseline. In preferred embodiments, the treated patient achieves a PRR defined as: 1) an eGFR within the normal range or no less than 85% of baseline, and 2)≥50% reduction in 24-hour UPCR to sub-nephrotic level compared to baseline

Success of treatment overtime may be measured by various techniques and surveys, including assessment of CRR, PRR, steroid reduction, eGFR, Urine Albumin-to-Creatinine Ratio (UACR), UPCR, FACIT-Fatigue score (Cella et al (1993) J. Clin. Oncol; 11(3):570-9, Yellen et al (1997) J Pain Symptom Manage; 13(2):63-74), Short Form Health Survey (SF-36) (Holloway et al (2014) Health Qual Life Outcomes; 12:116), Medical Outcome Short Form Health Survey (SF-36 Physical Component Summary (PCS)) (Ware et al (1994) SF-36 Health Survey manual and interpretation guide. Update. Boston: The Health Institute, New England Medical Center), LupusQoL (Yazdany (2011) Arthritis Care Res 63(11): S413-9), improvement in multiple lupus domains, e.g., SLEDAI-2000 (Bombardier et al (1992) 35(6):630-40), CLASI (Albrecht et al (2005) J. Invest. Dermatol; 125:889-94), DAS-28 (Ceccarelli et al (2014) Scientific World Journal; article ID: 236842; Cipriano (2015) Reumatismo; 62(2):62-7), LLDAS (Franklyn et al (2016) Ann. Rheum. Dis; 75(9):1615-21).

As used herein, the term “baseline” and the like (e.g., “baseline value”) refer to the value of a given variable prior to a subject being treated, e.g., with a disclosed IL-17 antibody or antigen-binding fragment thereof (e.g., secukinumab).

As used herein, the phrase “inactive urinary sediment” is a measure referring to a urine test, typically undertaken by centrifuging urine to concentrate substances, wherein there are ≤5 red blood cells and/or white blood cells per high power field (hpf). See, e.g., Cavanaugh and Perazella (2019) Am J. Kid. Diseases. 73(2):258-72.

As used herein, the phrase “cellular cast” refers to small tube-shaped particles made of cells (e.g., white blood cells, red blood cells, kidney cells) that can be found when urine is examined under the microscope during urinalysis. See, e.g., Ringsrud (2001) “Casts in the Urine Sediment” Laboratory Medicine (4)32.

In some embodiments, the patient is an adult human patient having LN. Is some embodiments, the patient is a pediatric human patient having LN. The upper age limit used to define a pediatric patient varies among experts, and can include adolescents up to the age of 21 (see, e.g., Berhman et a. (1996) Nelson Textbook of Pediatrics, 15th Ed. Philadelphia: W.B. Saunders Company; Rudolph AM, et al. (2002) Rudolph's Pediatrics, 21st Ed. New York: McGraw-Hill; and Avery(1994) First LR. Pediatric Medicine, 2nd Ed. Baltimore: Williams & Wilkins). As used herein, the term “Pediatric” generally refers to a human who is sixteen years old or younger, which is the definition of a pediatric human used by the US FDA.

In some embodiments, the pediatric patient is administered a SC dose of the IL-17 antibody (e.g., secukinumab) weekly during week 0, 1, 2, 3, and 4, and then every two weeks or four weeks (preferably every four weeks) thereafter as a dose of about 150 mg—about 300 mg (e.g., 150 mg or 300 mg), regardless of the patient's weight.

In some embodiments, the pediatric patient is administered a SC dose of the IL-17 antibody (e.g., secukinumab) weekly during week 0, 1, 2, 3, and 4, and then every two weeks or every four weeks thereafter as a dose of about 75 mg if the patient weighs<25 kg or about 150 mg if the patient weighs>25 kg. In some embodiments, the pediatric patient is administered a SC dose of the IL-17 antibody (e.g., secukinumab) weekly during week 0, 1, 2, 3, and 4, and then every two weeks or every four weeks thereafter as a dose of about 75 mg if the patient weighs<50 kg or about 150 mg if the patient weighs>50 kg.

In some embodiments, the pediatric patient is administered a SC dose of the IL-17 antibody (e.g., secukinumab) weekly during week 0, 1, 2, 3, and 4, and then every two weeks or every four weeks thereafter as a dose of about 150 mg if the patient weighs<25 kg or 300 mg if the patient weighs>25 kg. In some embodiments, the pediatric patient is administered a SC dose of the IL-17 antibody (e.g., secukinumab) weekly during week 0, 1, 2, 3, and 4, and then every two weeks or every four weeks thereafter as a dose of about 150 mg if the patient weighs<50 kg or 300 mg if the patient weighs>50 kg.

In some embodiments, the pediatric patient is administered an IV dose of the IL-17 antibody (e.g., secukinumab) of about 4 mg/kg—about 9 mg/kg (preferably about 6 mg/kg) once during week 0, and thereafter, as an IV dose of about 2—about 4 mg/kg (preferably about 3 mg/kg) every 4 weeks (monthly), beginning during week 4.

The IL-17 antagonists, e.g., IL-17 binding molecules (e.g., IL-17 antibody or antigen-binding fragment thereof, e.g., secukinumab) or IL-17 receptor binding molecules (e.g., IL-17 antibody or antigen-binding fragment thereof), may be used as a pharmaceutical composition when combined with a pharmaceutically acceptable carrier. Such a composition may contain, in addition to an IL-17 antagonist, carriers, various diluents, fillers, salts, buffers, stabilizers, solubilizers, and other materials known in the art. The characteristics of the carrier will depend on the route of administration. The pharmaceutical compositions for use in the disclosed methods may also contain additional therapeutic agents for treatment of the particular targeted disorder. For example, a pharmaceutical composition may also include anti-inflammatory agents. Such additional factors and/or agents may be included in the pharmaceutical composition to produce a synergistic effect with the IL-17 binding molecules, or to minimize side effects caused by the IL-17 antagonists, e.g., IL-17 binding molecules (e.g., IL-17 antibody or antigen-binding fragment thereof, e.g., secukinumab) or IL-17 receptor binding molecules (e.g., IL-17 antibody or antigen-binding fragment thereof). In preferred embodiments, the pharmaceutical compositions for use in the disclosed methods comprise secukinumab at 150 mg/ml.

Pharmaceutical compositions for use in the disclosed methods may be manufactured in conventional manner. In one embodiment, the pharmaceutical composition is provided in lyophilized form. For immediate administration it is dissolved in a suitable aqueous carrier, for example sterile water for injection or sterile buffered physiological saline. A reconstituted lyophilisate is referred to as a “reconstituent”. If it is considered desirable to make up a solution of larger volume for administration by infusion rather than a bolus injection, may be advantageous to incorporate human serum albumin or the patient's own heparinized blood into the saline at the time of formulation. The presence of an excess of such physiologically inert protein prevents loss of antibody by adsorption onto the walls of the container and tubing used with the infusion solution. If albumin is used, a suitable concentration is from 0.5 to 4.5% by weight of the saline solution. Other formulations comprise ready-to-use liquid formulations.

Antibodies, e.g., antibodies to IL-17, are typically formulated either in ready-to-use aqueous forms for parenteral administration or as lyophilisates for reconstitution with a suitable diluent prior to administration. In preferred embodiments of the disclosed methods and uses, the IL-17 antagonist, e.g., IL-17 antibody, e.g., secukinumab, is formulated as ready-to-use (i.e., a stable ready-to-use) liquid pharmaceutical formulation. In some embodiments of the disclosed methods and uses, the IL-17 antagonist, e.g., IL-17 antibody, e.g., secukinumab, is formulated as a lyophilisate. Suitable lyophilisate formulations can be reconstituted in a small liquid volume (e.g., 2 mL or less, e.g., 2 mL, 1 mL, etc.) to allow subcutaneous administration and can provide solutions with low levels of antibody aggregation. The use of antibodies as the active ingredient of pharmaceuticals is now widespread, including the products HERCEPTIN™ (trastuzumab), RITUXAN™ (rituximab), SYNAGIS™ (palivizumab), etc. Techniques for purification of antibodies to a pharmaceutical grade are known in the art. When a therapeutically effective amount of an IL-17 antagonist, e.g., IL-17 binding molecules (e.g., IL-17 antibody or antigen-binding fragment thereof, e.g., secukinumab) or IL-17 receptor binding molecules (e.g., IL-17 antibody or antigen-binding fragment thereof) is administered by intravenous, cutaneous or subcutaneous injection, the IL-17 antagonist will be in the form of a pyrogen-free, parenterally acceptable solution. A pharmaceutical composition for intravenous, cutaneous, or subcutaneous injection may contain, in addition to the IL-17 antagonist, an isotonic vehicle such as sodium chloride, Ringer's solution, dextrose, dextrose and sodium chloride, lactated Ringer's solution, or other vehicle as known in the art. A preferred lyophilisate formulation of secukinumab is disclosed in PCT Publication WO2012059598, which is incorporated by reference as it relates to this formulation. Preferred liquid ready-to-use formulations of secukinumab are disclosed in PCT Publication WO2016103153, which is incorporated by reference in its entirety.

In practicing some of the methods of treatment or uses of the present disclosure, a therapeutically effective amount of an IL-17 antagonist, e.g., IL-17 binding molecule (e.g., IL-17 antibody or antigen-binding fragment thereof, e.g., secukinumab) or IL-17 receptor binding molecule (e.g., IL-17 antibody or antigen-binding fragment thereof) is administered to a patient, e.g., a mammal (e.g., a human). While it is understood that the disclosed methods provide for treatment of LN patients using an IL-17 antagonist (e.g., secukinumab), this does not preclude that, if the patient is to be ultimately treated with an IL-17 antagonist, such IL-17 antagonist therapy is necessarily a monotherapy. Indeed, if a patient is selected for treatment with an IL-17 antagonist, then the IL-17 antagonist (e.g., secukinumab) may be administered in accordance with the methods of the disclosure either alone or in combination with other agents and therapies for treating LN patients, e.g., in combination with at least one additional LN agent. When co-administered with one or more additional LN agent(s), an IL-17 antagonist may be administered either simultaneously with the other agent, or sequentially. If administered sequentially, the attending physician will decide on the appropriate sequence of administering the IL-17 antagonist in combination with other agents and the appropriate dosages for co-delivery.

Various therapies may be beneficially combined with the disclosed IL-17 antibodies, such as secukinumab, during treatment of LN. Non-limiting examples of LN agents used in systemic treatment with the disclosed IL-17 antibodies, such as secukinumab, include further IL-17 antagonists (ixekizumab, brodalumab, CJM112), steroids (e.g., corticosteroids, e.g., glucocorticoids, e.g., prednisolone, prednisone, methylprednisolone, etc.), e.g., mycophenolate mofetil (MMF), cyclosporine A, rituximab, ocrelizumab, abatacept, azathioprine (AZA), calcineurin inhibitors, cyclosporine A, tacrolimus, cyclophosphamide (CYC), mycophenolic acid (MPA) (including salts thereof), voclosporin, belimumab, ustekinumab, iguratimod, anifrolumab, BI655064, CFZ533, and combination thereof. Preferred LN agents for use in the disclosed kits, methods, and uses with the IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) are corticosteroids (e.g., glucocorticoids, e.g., methylprednisolone, prednisolone, prednisone), mycophenolate mofetil (MMF), mycophenolic acid (MPA) (including salts thereof) (collectively “MPA”), cyclophosphamide (CYC), and combinations thereof.

A skilled artisan will be able to discern the appropriate dosages of the above LN agents for co-delivery with the disclosed IL-17 antibodies, such as secukinumab. See, e.g., Hahn et al. (2012) Arthritis Care Res (Hoboken) 64(6): 797-808.

An IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) is conveniently administered parenterally, e.g., intravenously (e.g., into the antecubital or other peripheral vein), intramuscularly, or subcutaneously. The duration of intravenous (IV) therapy using a pharmaceutical composition of the present disclosure will vary, depending on the severity of the disease being treated and the condition and personal response of each individual patient. Also contemplated is subcutaneous (SC) therapy using a pharmaceutical composition of the present disclosure. The health care provider will decide on the appropriate duration of IV or SC therapy and the timing of administration of the therapy, using the pharmaceutical composition of the present disclosure. In preferred embodiments, the IL-17 antagonist (e.g., secukinumab) is administered via the subcutaneous (SC) route.

The IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient SC, e.g., at about 150 mg—about 300 mg (e.g., about 150 mg, about 300 mg) weekly during weeks 0, 1, 2, 3, and 4, and thereafter administered to the patient SC, e.g., at about 150 mg—about 300 mg (e.g., about 150 mg, about 300 mg) monthly (every 4 weeks), beginning during week 8. In this manner, the patient is dosed SC with about 150 mg—about 300 mg (e.g., about 150 mg or about 300 mg) of the IL-17 antagonist (e.g., secukinumab) during weeks 0, 1, 2, 3, 4, 8, 12, 16, 20, etc.

Alternatively, the IL-17 antagonist (e.g., IL-17 antibody or antigen-binding fragment thereof, such as secukinumab) may be administered to the patient intravenously (IV). Preferred IV regimens (dose and administration scheme) for use with the disclosed IL-17 antagonists to treat LN are provided in Table 2.

TABLE 2
Preferred IV/IV regimens for use in the disclosed methods employing an IL-17
antagonist, e.g., IL-17 binding molecule (e.g., IL-17 antibody or antigen-
binding fragment thereof, e.g., secukinumab) or IL-17 receptor binding molecule
(e.g., IL-17 receptor antibody or antigen-binding fragment thereof).

| Loading regimen (IV) | Maintenance regimen (IV) |
|---|---|
| about 4.0 mg/kg (e.g., 4.0 mg/kg) once during | about 2.0 mg/kg (e.g., 2.0 mg/kg) monthly |
| week 0 | (every 4 weeks), beginning during week 4 |
| about 4.0 mg/kg (e.g., 4.0 mg/kg) once during | about 3.0 mg/kg (e.g., 3.0 mg/kg) monthly |
| week 0 | (every 4 weeks), beginning during week 4 |
| about 4.0 mg/kg (e.g., 4.0 mg/kg) once during | about 4.0 mg/kg (e.g., 4.0 mg/kg) monthly |
| week 0 | (every 4 weeks), beginning during week 4 |
| about 5.0 mg/kg (e.g., 5.0 mg/kg) once during | about 2.5 mg/kg (e.g., 2.5 mg/kg) monthly |
| week 0 | (every 4 weeks), beginning during week 4 |
| about 6.0 mg/kg (e.g., 6.0 mg/kg) once during | about 2.0 mg/kg (e.g., 2.0 mg/kg) monthly |
| week 0 | (every 4 weeks), beginning during week 4 |
| about 6.0 mg/kg (e.g., 6.0 mg/kg) once during | about 3.0 mg/kg (e.g., 3.0 mg/kg) monthly |
| week 0 | (every 4 weeks), beginning during week 4 |
| about 6.0 mg/kg (e.g., 6.0 mg/kg) once during | about 4.0 mg/kg (e.g., 4.0 mg/kg) monthly |
| week 0 | (every 4 weeks), beginning during week 4 |
| about 7.0 mg/kg (e.g., 7.0 mg/kg) once during | about 3.5 mg/kg (e.g., 3.5 mg/kg) monthly |
| week 0 | (every 4 weeks), beginning during week 4 |
| about 8.0 mg/kg (e.g., 8.0 mg/kg) once during | about 4.0 mg/kg (e.g., 4.0 mg/kg) monthly |
| week 0 | (every 4 weeks), beginning during week 4 |
| about 9.0 mg/kg (e.g., 9.0 mg/kg) once during | about 2.0 mg/kg (e.g., 2.0 mg/kg) monthly |
| week 0 | (every 4 weeks), beginning during week 4 |
| about 9.0 mg/kg (e.g., 9.0 mg/kg) once during | about 3.0 mg/kg (e.g., 3.0 mg/kg) monthly |
| week 0 | (every 4 weeks), beginning during week 4 |
| about 9.0 mg/kg (e.g., 9.0 mg/kg) once during | about 4.0 mg/kg (e.g., 4.0 mg/kg) monthly |
| week 0 | (every 4 weeks), beginning during week 4 |
| about 10 mg/kg (e.g., 10 mg/kg) monthly | about 10 mg/kg (e.g., 10 mg/kg) every two |
| (every 4 weeks) during week 0, 4, 8 | months (every 8 weeks), beginning during |
| | week 16 |

In some embodiments, it is contemplated that the IL-17 antagonist (e.g., IL-17 antibody or antigen-binding fragment thereof, such as secukinumab) may be administered to the patient intravenously (IV) at a dose of about 4 mg/kg—about 9 mg/kg (preferably about 6 mg/kg) once during week 0, and thereafter, as an IV dose of about 2—about 4 mg/kg (preferably about 3 mg/kg) every 4 weeks (monthly), beginning during week 4. In this manner, the patient is dosed IV with about 4 mg/kg—about 9 mg/kg (e.g., about 6 mg/kg) of the IL-17 antagonist (e.g., secukinumab) during weeks 0, 4, 8, 12, 16, 20, etc. In a preferred embodiment, the IL-17 antagonist (e.g., IL-17 antibody or antigen-binding fragment thereof, such as secukinumab) is administered to the patient intravenously (IV) at a dose of about 6 mg/kg once during week 0, and thereafter, as an IV dose of about 3 mg/kg every 4 weeks (monthly), beginning during week 4. In this manner, the patient is dosed IV with about 6 mg/kg of the IL-17 antagonist (e.g., secukinumab) during weeks 0, and thereafter, as an IV dose of about 3 mg/kg during week 4, 8, 12, 16, 20, etc.

In some embodiments, the IL-17 antagonist (e.g., IL-17 antibody or antigen-binding fragment thereof, such as secukinumab) is administered to the patient intravenously (IV) at a dose of about 4 mg/kg—about 9 mg/kg (preferably about 6 mg/kg) once during week 0, and thereafter, an IV dose of about 2.0— about 4 mg/kg (preferably about 3 mg/kg) every 8 weeks (every other month), beginning during week 4.

In some embodiments, it is contemplated that the IL-17 antagonist (e.g., IL-17 antibody or antigen-binding fragment thereof, such as secukinumab) may be administered to the patient intravenously (IV) at a dose of about 10 mg/kg monthly (every 4 weeks). In some embodiments, it is contemplated that the IL-17 antagonist (e.g., IL-17 antibody or antigen-binding fragment thereof, such as secukinumab) may be administered to the patient intravenously (IV) at a dose of about 10 mg/kg every two months (every 8 weeks). In some embodiments, it is contemplated that the IL-17 antagonist (e.g., IL-17 antibody or antigen-binding fragment thereof, such as secukinumab) may be administered to the patient intravenously (IV) at a dose of about 10 mg/kg monthly (every 4 weeks) during week 0, 4, 8, and thereafter at a dose of about 10 mg/kg (e.g., 10 mg/kg) every two months (every 8 weeks), beginning during week 16.

Alternatively, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient without a loading regimen, e.g., the antagonist may be administered to the patient SC at about 150 mg—about 300 mg (e.g., about 150 mg, about 300 mg) every two, four or eight weeks (preferably every four weeks). When dosed every four weeks, the patient receives drug, e.g., about 150 mg—about 300 mg (e.g., about 150 mg, about 300 mg) of the IL-17 antagonist (e.g., secukinumab), during weeks 0, 4, 8, 12, 16, 20, etc.

Alternatively, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient without a loading regimen, e.g., the antagonist may be administered to the patient IV at about 2.5 —about 4 mg/kg (preferably about 3 mg/kg) every month or at about 2.5— about 4 mg/kg (preferably about 3 mg/kg) every two months.

Alternatively, the IL-17 antagonists, e.g., IL-17 antibodies, e.g., secukinumab, can also be delivered orally (e.g., into the intestinal lumen using Rani Therapeutics technology, e.g., technology set forth in U.S. Pat. Nos. 8,734,429; 9,492,378; 9,456,988; 9,415,004; 9,6297,99; 9,757,548; 9,757,514; 9,402,806; US Pub. Appln. 2017/0189659, 2017/0100459).

It will be understood that dose escalation may be required for certain patients, e.g., LN patients that display inadequate response (e.g., as measured by any of the LN scoring systems disclosed herein, e.g., CRR, PRR, estimated glomerular filtration rate (eGFR), 24-hour urinary protein to creatinine ratio, Functional Assessment of Chronic Illness Therapy—Fatigue (FACIT-Fatigue©), Short Form Health Survey (SF-36 Physical Component Summary (PCS), Lupus Quality of Life (LupusQoL), etc.) to treatment with the IL-17 antagonists, e.g., IL-17 binding molecules (e.g., IL-17 antibody or antigen-binding fragment thereof, e.g., secukinumab) or IL-17 receptor binding molecules (e.g., IL-17 receptor antibody or antigen-binding fragment thereof) by week 10, week 12, week 14, week 16, week 18, week 20, week 22, week 24, week 48, week 52, or week 104 of treatment. Thus, SC dosages of secukinumab may be greater than about 150 mg —about 300 mg SC, e.g., about 200 mg, about 250 mg (in the case of an original 150 mg dose), about 350 mg, about 450 mg (in the case of an original 300 mg dose), etc.; similarly, IV dosages may be greater than about 2 mg/kg—about 9 mg/kg, e.g., about 2.5 mg/kg, about 3 mg/kg, 4 mg/kg, about 5 mg/kg, about 6 mg/kg (e.g., in the case of an original 2 mg/kg dose), about 9.5 mg/kg, 10 mg/kg, 11 mg/kg, 12 mg/kg, 15 mg/kg, 20 mg/kg, 25 mg/kg, 30 mg/kg, 35 mg/kg (in the case of an original 9 mg/kg mg dose), etc.

Similarly, more frequent dosing may be used during the maintenance regimen in certain patients, e.g., a patient having an inadequate response (e.g., partial response, failed response, or loss of response over time) to treatment with the IL-17 antibody or antigen-binding fragment thereof, e.g., secukinumab. These patients may be switched to more frequent administration (rather than increased dose), e.g., switched from administration of the IL-17 antibody or antigen-binding fragment thereof, e.g., secukinumab, every 4 weeks (monthly; Q4w) to administration every two weeks (Q2w) or every week (Q1w), or from administration every 2 weeks (Q2w) to administration every week (Q1w). This switch may be done as determined necessary by a physician, e.g., at week 10, week 12, week 14, week 16, week 18, week 20, week 22, week 24, week 48, week 52, or week 104 of treatment.

It will also be understood that dose reduction may also be used for certain patients, e.g., LP (e.g., CLP, MLP, LLP) patients that display a particularly robust treatment response, or an adverse event/response to treatment with the IL-17 antagonist (e.g., IL-17 antibody or antigen-binding fragment thereof, e.g., secukinumab). Thus, dosages of the IL-17 antagonist (e.g., IL-17 antibody or antigen-binding fragment thereof, e.g., secukinumab), may be lowered to less than about 150 mg—about 300 mg SC, e.g., about 250 mg, about 200 mg, about 150 mg (in the case of an original 300 mg dose); about 100 mg, about 50 mg (in the case of an original 150 mg dose), etc. Similarly, IV dosages may be lowered to less than about 8 mg/kg, e.g., about 7 mg/kg, 5 mg/kg, 4 mg/kg, 3 mg/kg, 2 mg/kg, 1 mg/kg, etc. In some embodiments, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient at an initial dose of 300 mg or 150 mg delivered SC, and the dose is then escalated to about 450 mg (in the case of an original 300 mg dose) or about 300 mg (in the case of an original 150 mg dose) if needed, as determined by a physician.

Similarly, less frequent dosing may be used during the maintenance regimen in certain patients, e.g., a patient having a particularly robust treatment response, or an adverse event/response to treatment with the IL-17 antibody or antigen-binding fragment thereof, e.g., secukinumab. These patients may be switched to less frequent administration (rather than decreased dose), e.g., switched from administration of the IL-17 antibody or antigen-binding fragment thereof, e.g., secukinumab, every 4 weeks (monthly; Q4w) to administration every six weeks (Q6w) or eight weeks (Q8w), or from administration of the IL-17 antibody or antigen-binding fragment thereof, e.g., secukinumab, every 2 weeks (monthly; Q2w) to administration every four weeks (Q4w) or every six weeks (Q6w). This switch may be done as determined necessary by a physician, e.g., at week 10, week 12, week 14, week 16, week 18, week 20, week 22, week 24, week 48, week 52, or week 104 of treatment.

As used herein, “fixed dose” refers to a flat dose, i.e., a dose that is unchanged regardless of a patient's characteristics. Thus, a fixed dose differs from a variable dose, such as a body-surface area-based dose or a weight-based dose (typically given as mg/kg). In some embodiments of the disclosed methods, uses, pharmaceutical compositions, kits, etc., the LN patient is administered fixed doses of the IL-17 antibody, e.g., fixed doses of secukinumab, e.g., fixed doses of about 75 mg—about 450 mg secukinumab, e.g., about 75 mg, about 150 mg, about 300 mg, about 400 mg or about 450 mg secukinumab. Alternatively, in some embodiments, the patient is administered a weight-based dose, e.g., a dose given in mg based on patient weight in kg (mg/kg).

The timing of dosing is generally measured from the day of the first dose of secukinumab (which is also known as “baseline”). However, health care providers often use different naming conventions to identify dosing schedules, as shown in Table 3.

TABLE 2
Common naming conventions for dosing regimens.

| Week | 0/1 | 1/2 | 2/3 | 3/4 | 4/5 | 5/6 | 6/7 | 7/8 | 8/9 | 9/10 | 10/11 | etc |
|---|---|---|---|---|---|---|---|---|---|---|---|---|
| 1^{st} day of week | 0/1 | 7/8 | 14/15 | 21/22 | 28/29 | 35/36 | 42/43 | 49/50 | 56/57 | 63/64 | 70/71 | etc. |

Bolded items refer to the naming convention used herein.

Notably, week zero may be referred to as week one by some health care providers, while day zero may be referred to as day one by some health care providers. Thus, it is possible that different physicians will designate, e.g., a dose as being given during week 3/on day 21, during week 3/on day 22, during week 4/on day 21, during week 4/on day 22, while referring to the same dosing schedule. For consistency, the first week of dosing will be referred to herein as week 0, while the first day of dosing will be referred to as day 1. However, it will be understood by a skilled artisan that this naming convention is simply used for consistency and should not be construed as limiting, i.e., weekly dosing is the provision of a weekly dose of the IL-17 antibody regardless of whether the physician refers to a particular week as “week 1” or “week 2”.

In a one dosing regimen, the antibody is administered during week 0, 1, 2, 3, 4, 8, 12, 16, 20, etc. Some providers may refer to this regimen as weekly for five weeks and then monthly (or every 4 weeks) thereafter, beginning during week 8, while others may refer to this regimen as weekly for four weeks and then monthly (or every 4 weeks) thereafter, beginning during week 4. It will be appreciated by a skilled artisan that administering a patient an injection at weeks 0, 1, 2 and 3, followed by once monthly dosing starting at week 4 is the same as: 1) administering the patient an injection at weeks 0, 1, 2, 3, and 4, followed by once monthly dosing starting at week 8; 2) administering the patient an injection at weeks 0, 1, 2, 3 and 4 followed by dosing every 4 weeks; and 3) administering the patient an injection at weeks 0, 1, 2, 3 and 4 followed by monthly administration.

In one embodiment, the antibody is administered to an LN patient during week 0, 1, 2, 3, 4, 6, 8, 10, 12, etc. Some providers may refer to this regimen as weekly for five weeks and then every other week (or every 2 weeks) thereafter, beginning during week 6, while others may refer to this regimen as weekly for four weeks and then every other week (or every 2 weeks) thereafter, beginning during week 4. It will be appreciated by a skilled artisan that administering a patient an injection at weeks 0, 1, 2 and 3, followed by administration every other week (or every 2 weeks) starting at week 4 is the same as: 1) administering the patient an injection at weeks 0, 1, 2, 3, and 4, followed by dosing every other week (or every 2 weeks) starting at week 6; 2) administering the patient an injection at weeks 0, 1, 2, 3 and 4 followed by dosing every 2 weeks; and 3) administering the patient an injection at weeks 0, 1, 2, 3 and 4 followed by every other week administration.

As used herein, the phrase “formulated at a dosage to allow [route of administration] delivery of [a designated dose]” is used to mean that a given pharmaceutical composition can be used to provide a desired dose of an IL-17 antagonist, e.g., an IL-17 antibody, e.g., secukinumab, via a designated route of administration (e.g., SC or IV). As an example, if a desired SC dose is 300 mg, then a clinician may use 2 ml of an IL-17 antibody formulation having a concentration of 150 mg/ml, 1 ml of an IL-17 antibody formulation having a concentration of 300 mg/ml, 0.5 ml of an IL-17 antibody formulation having a concentration of 600 mg/ml, etc. In each such case, these IL-17 antibody formulations are at a concentration high enough to allow subcutaneous delivery of the IL-17 antibody. Subcutaneous delivery typically requires delivery of volumes of less than or equal to about 2 ml, preferably a volume of about 1 ml or less. Preferred formulations are ready-to-use liquid pharmaceutical compositions comprising about 25 mg/mL to about 150 mg/mL secukinumab, about 10 mM to about 30 mM histidine pH 5.8, about 200 mM to about 225 mM trehalose, about 0.02% polysorbate 80, and about 2.5 mM to about 20 mM methionine.

As used herein, the phrase “container having a sufficient amount of the IL-17 antagonist to allow delivery of [a designated dose]” is used to mean that a given container (e.g., vial, pen, syringe) has disposed therein a volume of an IL-17 antagonist (e.g., as part of a pharmaceutical composition) that can be used to provide a desired dose. As an example, if a desired dose is 300 mg, then a clinician may use 2 mL from a container that contains an IL-17 antibody formulation with a concentration of 150 mg/mL, 1 mL from a container that contains an IL-17 antibody formulation with a concentration of 300 mg/mL, 0.5 mL from a container contains an IL-17 antibody formulation with a concentration of 600 mg/ml, etc. In each such case, these containers have a sufficient amount of the IL-17 antagonist to allow delivery of the desired 300 mg dose.

In some embodiments of the disclosed uses, methods, and kits, the dose of the IL-17 antibody (e.g., secukinumab) or an antigen-binding fragment thereof is about 300 mg, the IL-17 antibody (e.g., secukinumab) or an antigen-binding fragment thereof is comprised in a liquid pharmaceutical formulation at a concentration of 150 mg/ml, and 2 ml of the pharmaceutical formulation is disposed within two pre-filled syringes, injection pens, or autoinjectors, each having 1 ml of the pharmaceutical formulation. In this case, the patient receives two injections of 1 ml each, for a total dose of 300 mg, during each administration. In some embodiments, the dose of the IL-17 antibody (e.g., secukinumab) or an antigen-binding fragment thereof is about 300 mg, the IL-17 antibody (e.g., secukinumab) or an antigen-binding fragment thereof is comprised in a liquid pharmaceutical formulation at a concentration of 150 mg/ml, and 2 ml of the pharmaceutical formulation is disposed within an autoinjector or PFS. In this case, the patient receives one injection of 2 ml, for a total dose of 300 mg, during each administration. In methods employing one injection of 2 ml (e.g., via a single PFS or autoinjector) (i.e., a “single-dose preparation”), the drug exposure (AUC) and maximal concentration (Cₘₐₓ) is equivalent (similar to, i.e., within the range of acceptable variation according to US FDA standards) to methods employing two injections of 1 ml (e.g., via two PFSs or two AIs) (i.e., a “multiple-dose preparation”).

Accordingly, disclosed herein are methods of treating LN, comprising subcutaneously (SC) administering to a patient in need thereof a dose of about 150 mg—about 300 mg of an IL-17 antibody (e.g., secukinumab) or an antigen-binding fragment thereof, weekly during weeks 0, 1, 2, 3, and 4, and thereafter SC at a dose of about 150 mg—about 300 mg monthly (every 4 weeks), beginning during week 8, wherein the IL-17 antibody or an antigen-binding fragment thereof binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody has a K_{D} of about 100-200 pM as measured by a biosensor system (e.g., BIACORE®) or surface plasmon resonance, and wherein the IL-17 antibody has an in vivo half-life of about 23 to about 30 days. Also disclosed herein is an IL-17 antibody (e.g. secukinumab) or an antigen-binding fragment thereof, for use in treating LN, which is to be subcutaneously (SC) administered to a patient in need thereof at a dose of about 150 mg—about 300 mg weekly during weeks 0, 1, 2, 3, and 4, and thereafter SC at a dose of about 150 mg —about 300 mg monthly (every 4 weeks), beginning during week 8, wherein the IL-17 antibody or an antigen-binding fragment thereof binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody has a K_{D} of about 100-200 pM as measured by a biosensor system (e.g., BIACORE®) or surface plasmon resonance, and wherein the IL-17 antibody has an in vivo half-life of about 23 to about 30 days. Alternatively, disclosed herein is an IL-17 antibody (e.g. secukinumab) or an antigen-binding fragment thereof, for use in the manufacture of a medicament for treating LN, which is to be subcutaneously (SC) administered to a patient in need thereof at a dose of about 150 mg—about 300 mg of the IL-17 antibody or an antigen-binding fragment thereof, weekly during weeks 0, 1, 2, 3, and 4, and thereafter SC at a dose of about 150 mg—about 300 mg monthly (every 4 weeks), beginning during week 8, wherein the IL-17 antibody or an antigen-binding fragment thereof binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody has a K_{D} of about 100-200 pM as measured by a biosensor system (e.g., BIACORE®) or surface plasmon resonance, and wherein the IL-17 antibody has an in vivo half-life of about 23 to about 30 days.

Disclosed herein are methods of treating LN, comprising subcutaneously (SC) administering to a patient in need thereof a dose of about 150 mg—about 300 mg (e.g., about 150 mg, about 300 mg) of an IL-17 antibody (e.g., secukinumab) or an antigen-binding fragment thereof, weekly during weeks 0, 1, 2, 3, and 4, and thereafter SC at a dose of about 150 mg to about 300 mg (e.g., about 150 mg, about 300 mg) monthly (every 4 weeks), beginning during week 8. Also disclosed herein is an IL-17 antibody (e.g. secukinumab) or an antigen-binding fragment thereof, for use in treating LN, which is to be subcutaneously (SC) administered to a patient in need thereof at a dose of about 150 mg to about 300 mg (e.g., about 150 mg, about 300 mg) weekly during weeks 0, 1, 2, 3, and 4, and thereafter SC at a dose of about 150 mg to about 300 mg (e.g., about 150 mg, about 300 mg) monthly (every 4 weeks), beginning during week 8. Also disclosed herein is an IL-17 antibody (e.g. secukinumab) or an antigen-binding fragment thereof, for use in the manufacture of a medicament for treating LN, which is to be subcutaneously (SC) administered to a patient in need thereof at a dose of about 150 mg to about 300 mg (e.g., about 150 mg, about 300 mg) of the IL-17 antibody or an antigen-binding fragment thereof, weekly during weeks 0, 1, 2, 3, and 4, and thereafter SC at a dose of about 150 mg to about 300 mg (e.g., about 150 mg, about 300 mg) monthly (every 4 weeks), beginning during week 8.

Disclosed herein are methods of treating LN, comprising subcutaneously (SC) administering to a patient in need thereof a dose of about 150 mg—about 300 mg of an IL-17 antibody (e.g., secukinumab) or an antigen-binding fragment thereof, weekly during weeks 0, 1, 2, 3, and 4, and thereafter SC at a dose of about 150 mg—about 300 mg every 2 weeks, beginning during week 6, wherein the IL-17 antibody or antigen-binding fragment thereof comprises: i) an immunoglobulin V_{H} domain comprising the amino acid sequence set forth as SEQ ID NO:8 and an immunoglobulin V_{L} domain comprising the amino acid sequence set forth as SEQ ID NO:10; ii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO: 5 and SEQ ID NO:6; or iii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO: 5 and SEQ ID NO:6. Also disclosed herein is an IL-17 antibody (e.g. secukinumab) or an antigen-binding fragment thereof, for use in treating LN, which is to be subcutaneously (SC) administering to a patient in need thereof at a dose of about 150 mg—about 300 mg of the IL-17 antibody (e.g. secukinumab) or an antigen-binding fragment thereof, weekly during weeks 0, 1, 2, 3, and 4, and thereafter SC at a dose of about 150 mg—about 300 mg every 2 weeks, beginning during week 6, wherein the IL-17 antibody or an antigen-binding fragment thereof comprises: i) an immunoglobulin VH domain comprising the amino acid sequence set forth as SEQ ID NO: 8 and an immunoglobulin VL domain comprising the amino acid sequence set forth as SEQ ID NO:10; ii) an immunoglobulin VH domain comprising the hypervariable regions set forth as SEQ ID NO:1, SEQ ID NO:2, and SEQ ID NO:3 and an immunoglobulin VL domain comprising the hypervariable regions set forth as SEQ ID NO:4, SEQ ID NO:5 and SEQ ID NO:6; or iii) an immunoglobulin VH domain comprising the hypervariable regions set forth as SEQ ID NO:11, SEQ ID NO:12 and SEQ ID NO:13 and an immunoglobulin VL domain comprising the hypervariable regions set forth as SEQ ID NO:4, SEQ ID NO:5 and SEQ ID NO:6. Also disclosed herein is an IL-17 antibody (e.g. secukinumab) or an antigen-binding fragment thereof, for use in the manufacture of a medicament for treating LN, which is to be subcutaneously (SC) administering to a patient in need thereof at a dose of about 150 mg—about 300 mg of the IL-17 antibody or an antigen-binding fragment thereof (e.g. secukinumab), weekly during weeks 0, 1, 2, 3, and 4, and thereafter SC at a dose of about 150 mg—about 300 mg every 2 weeks, beginning during week 6, wherein the IL-17 antibody or an antigen-binding fragment thereof comprises: i) an immunoglobulin VH domain comprising the amino acid sequence set forth as SEQ ID NO:8 and an immunoglobulin VL domain comprising the amino acid sequence set forth as SEQ ID NO:10; ii) an immunoglobulin VH domain comprising the hypervariable regions set forth as SEQ ID NO:1, SEQ ID NO:2, and SEQ ID NO:3 and an immunoglobulin VL domain comprising the hypervariable regions set forth as SEQ ID NO:4, SEQ ID NO: 5 and SEQ ID NO:6; or iii) an immunoglobulin VH domain comprising the hypervariable regions set forth as SEQ ID NO:11, SEQ ID NO:12 and SEQ ID NO:13 and an immunoglobulin VL domain comprising the hypervariable regions set forth as SEQ ID NO:4, SEQ ID NO: 5 and SEQ ID NO:6.

Disclosed herein are methods of treating LN, comprising subcutaneously (SC) administering to a patient in need thereof a dose of about 150 mg—about 300 mg (e.g., about 150 mg, about 300 mg) of an IL-17 antibody (e.g., secukinumab) or an antigen-binding fragment thereof, weekly during weeks 0, 1, 2, 3, and 4, and thereafter SC at a dose of about 150 mg to about 300 mg (e.g., about 150 mg, about 300 mg) every 2 weeks, beginning during week 6. Also disclosed herein is an IL-17 antibody (e.g. secukinumab) or an antigen-binding fragment thereof, for use in treating LN, which is to be subcutaneously (SC) administered to a patient in need thereof at a dose of about 150 mg to about 300 mg (e.g., about 150 mg, about 300 mg) weekly during weeks 0, 1, 2, 3, and 4, and thereafter SC at a dose of about 150 mg to about 300 mg (e.g., about 150 mg, about 300 mg) every 2 weeks, beginning during week 6. Also disclosed herein is an IL-17 antibody (e.g. secukinumab) or an antigen-binding fragment thereof, for use in the manufacture of a medicament for treating LN, which is to be subcutaneously (SC) administered to a patient in need thereof at a dose of about 150 mg to about 300 mg (e.g., about 150 mg, about 300 mg) of the IL-17 antibody or an antigen-binding fragment thereof, weekly during weeks 0, 1, 2, 3, and 4, and thereafter SC at a dose of about 150 mg to about 300 mg (e.g., about 150 mg, about 300 mg) every 2 weeks, beginning during week 6.

In preferred embodiments of the disclosed methods, uses and kits, the dose of the IL-17 antibody or antigen-binding fragment (e.g., secukinumab) is about 150 mg or about 300 mg.

In preferred embodiments of the disclosed methods, uses and kits, the IL-17 antibody or antigen-binding fragment thereof is administered weekly during weeks 0, 1, 2, 3, and 4, and thereafter every month (every four weeks). In this manner, the IL-17 antibody or antigen-binding fragment thereof (e.g., secukinumab) is administered during week 0, 1, 2, 3, 4, 8, 12, 16, etc.

In other embodiments of the disclosed methods, uses and kits, the IL-17 antibody or antigen-binding fragment thereof is administered weekly during weeks 0, 1, 2, 3, and 4, and thereafter every two weeks. In this manner, the IL-17 antibody or antigen-binding fragment thereof (e.g., secukinumab) is administered during week 0, 1, 2, 3, 4, 6, 8, 10, 12, 14, 16, etc.

Disclosed herein are methods of treating LN, comprising intravenously (IV) administering to a patient in need thereof a dose of about 4 mg/kg—about 9 mg/kg (preferably about 6 mg/kg) of an IL-17 antibody, or an antigen-binding fragment thereof, once during week 0, and thereafter administering an IV dose of about 2 mg/kg—about 4 mg/kg (preferably about 3 mg/kg) of the IL-17 antibody, or an antigen-binding fragment thereof every four weeks, beginning during week four, wherein the IL-17 antibody or antigen-binding fragment thereof comprises: i) an immunoglobulin V_{H} domain comprising the amino acid sequence set forth as SEQ ID NO:8 and an immunoglobulin V_{L} domain comprising the amino acid sequence set forth as SEQ ID NO:10; ii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO: 5 and SEQ ID NO:6; or iii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO: 5 and SEQ ID NO:6. Also disclosed herein is an IL-17 antibody (e.g., secukinumab) or an antigen-binding fragment thereof, for use in treating LN, which is to be intravenously (IV) administered to a patient in need thereof at a dose of about 4 mg/kg—about 9 mg/kg (preferably about 6 mg/kg) once during week 0, and thereafter at a dose of about 2 mg/kg—about 4 mg/kg (preferably about 3 mg/kg) every four weeks, beginning during week four, wherein the IL-17 antibody or antigen-binding fragment thereof comprises: i) an immunoglobulin VH domain comprising the amino acid sequence set forth as SEQ ID NO:8 and an immunoglobulin VL domain comprising the amino acid sequence set forth as SEQ ID NO:10; ii) an immunoglobulin VH domain comprising the hypervariable regions set forth as SEQ ID NO:1, SEQ ID NO:2, and SEQ ID NO:3 and an immunoglobulin VL domain comprising the hypervariable regions set forth as SEQ ID NO:4, SEQ ID NO:5 and SEQ ID NO:6; or iii) an immunoglobulin VH domain comprising the hypervariable regions set forth as SEQ ID NO:11, SEQ ID NO:12 and SEQ ID NO:13 and an immunoglobulin VL domain comprising the hypervariable regions set forth as SEQ ID NO:4, SEQ ID NO:5 and SEQ ID NO:6. Also disclosed herein is an IL-17 antibody (e.g. secukinumab) or an antigen-binding fragment thereof, for use in the manufacture of a medicament for treating LN, which is to be intravenously (IV) administered to a patient in need thereof at a dose of about 4 mg/kg—about 9 mg/kg (preferably about 6 mg/kg) once during week 0, and thereafter at a dose of about 2 mg/kg—about 4 mg/kg (preferably about 3 mg/kg) every four weeks, beginning during week four, wherein the IL-17 antibody or antigen-binding fragment thereof comprises: i) an immunoglobulin VH domain comprising the amino acid sequence set forth as SEQ ID NO: 8 and an immunoglobulin VL domain comprising the amino acid sequence set forth as SEQ ID NO:10; ii) an immunoglobulin VH domain comprising the hypervariable regions set forth as SEQ ID NO:1, SEQ ID NO:2, and SEQ ID NO:3 and an immunoglobulin VL domain comprising the hypervariable regions set forth as SEQ ID NO:4, SEQ ID NO: 5 and SEQ ID NO:6; or iii) an immunoglobulin VH domain comprising the hypervariable regions set forth as SEQ ID NO:11, SEQ ID NO:12 and SEQ ID NO:13 and an immunoglobulin VL domain comprising the hypervariable regions set forth as SEQ ID NO:4, SEQ ID NO: 5 and SEQ ID NO:6.

Disclosed herein are methods of treating LN, comprising intravenously (IV) administering to a patient in need thereof a dose of about 4 mg/kg—about 9 mg/kg (preferably about 6 mg/kg) of an IL-17 antibody (e.g., secukinumab) or an antigen-binding fragment thereof, once during week 0, and thereafter administering an IV dose of about 2 mg/kg—about 4 mg/kg (preferably about 3 mg/kg) of the IL-17 antibody (e.g., secukinumab), or an antigen-binding fragment thereof every four weeks, beginning during week four. Also disclosed herein is an IL-17 antibody (e.g. secukinumab) or an antigen-binding fragment thereof, for use in treating LN, which is to be intravenously (IV) administered to a patient in need thereof at a dose of about 4 mg/kg—about 9 mg/kg (preferably about 6 mg/kg) once during week 0, and thereafter at a dose of about 2 mg/kg—about 4 mg/kg (preferably about 3 mg/kg) every four weeks, beginning during week four. Also disclosed herein is an IL-17 antibody (e.g. secukinumab) or an antigen-binding fragment thereof, for use in the manufacture of a medicament for treating LN, which is to be intravenously (IV) administered to a patient in need thereof at a dose of about 4 mg/kg—about 9 mg/kg (preferably about 6 mg/kg) once during week 0, and thereafter at a dose of about 2 mg/kg—about 4 mg/kg (preferably about 3 mg/kg) every four weeks, beginning during week four.

In other embodiments of the disclosed methods, uses and kits, the initial IV dose of the IL-17 antibody or antigen-binding fragment (e.g., secukinumab) administered during week 0 is about 6 mg/kg and the monthly IV dose administered thereafter is about 3 mg/kg. In preferred embodiments, the IL-17 antibody or antigen-binding fragment thereof (e.g., secukinumab) is administered IV during week 0, 4, 8, 12, 16, etc.

In other embodiments of the disclosed methods, uses and kits, the IL-17 antibody or antigen-binding fragment thereof (e.g., secukinumab) is administered IV at a dose of about 3 mg/kg monthly during weeks 0, 4, and 8, and thereafter IV at a dose of about 3 mg/kg every two months (every eight weeks). In this manner, the IL-17 antibody or antigen-binding fragment thereof (e.g., secukinumab) is administered IV at a dose of about 3 mg/kg during month 0, 1, 2, 4, 6, 8, etc.

In other embodiments of the disclosed methods, uses and kits, the IL-17 antibody or antigen-binding fragment thereof (e.g., secukinumab) is administered IV at a dose of about 10 mg/kg monthly during weeks 0, 4, and 8, and thereafter IV at a dose of about 10 mg/kg every two months (every eight weeks). In this manner, the IL-17 antibody or antigen-binding fragment thereof (e.g., secukinumab) is administered IV at a dose of about 10 mg/kg during month 0, 1, 2, 4, 6, 8, etc.

In preferred embodiments of the disclosed methods, uses and kits, the patient achieves a complete renal response (CRR) by week 52 of treatment, a partial renal response (PPR) by week 52 of treatment, improvement in UPCR by week 52 of treatment, improvement in eGFR by week 52 of treatment, steroid reduction (e.g., to a dose of <11 mg daily) by week 52 of treatment, inactive urinary sediments (no cellular casts) by week 52 of treatment, improvement in FACIT-F fatigue score by week 52 of treatment, or any combination thereof.

In preferred embodiments of the disclosed methods, uses and kits, prior to treatment with the IL-17 antibody or antigen-binding fragment thereof (e.g., secukinumab), the patient was administered mycophenolic acid (MPA) or cyclophosphamide (CYC), and, optionally at least one steroid.

In preferred embodiments of the disclosed methods, uses and kits, prior to treatment with the IL-17 antibody or antigen-binding fragment thereof (e.g., secukinumab), the LN was inadequately controlled by the prior treatment with MPA or CYC, and, optionally the at least one steroid.

In preferred embodiments of the disclosed methods, uses and kits, during treatment with the IL-17 antibody or antigen-binding fragment thereof (e.g., secukinumab), the patient is concomitantly administered MPA or CYC, and, optionally at least one steroid.

In preferred embodiments of the disclosed methods, uses and kits, during treatment with the IL-17 antibody or antigen-binding fragment thereof (e.g., secukinumab), the dose of MPA or CYC administered to the patient is reduced, and wherein the patient does not experience a flare as a result of said reduction.

In preferred embodiments of the disclosed methods, uses and kits, during treatment with the IL-17 antibody or antigen-binding fragment thereof (e.g., secukinumab), the dose of the at least one steroid administered to the patient is reduced using a taper regimen, and wherein the patient does not experience a flare as a result of said reduction.

In preferred embodiments of the disclosed methods, uses and kits, the patient does not have concomitant plaque-type psoriasis.

In preferred embodiments of the disclosed methods, uses and kits, the patient has active LN.

In preferred embodiments of the disclosed methods, uses and kits, the patient has International Society of Nephrology/Renal Pathology Society (ISN/RPS) Class III or IV LN.

In preferred embodiments of the disclosed methods, uses and kits, the ISN/RPS Class III IN is not Class III(C).

In preferred embodiments of the disclosed methods, uses and kits, the ISN/RPS Class IV LN is not Class IV-S(C) or IV-G(C).

In preferred embodiments of the disclosed methods, uses and kits, the patient has features of ISN/RPS Class V LN.

In preferred embodiments of the disclosed methods, uses and kits, the patient is additionally administered at least one LN agent selected from the group consisting of rituximab, ocrelizumab, abatacept, azathioprine, a calcineurin inhibitor, cyclosporine A, tacrolimus, cyclophosphamide, mycophenolic acid, voclosporin, belimumab, ustekinumab, iguratimod, anifrolumab, BI655064, CFZ533, and combinations thereof.

In preferred embodiments of the disclosed methods, uses and kits, the patient is an adult.

In preferred embodiments of the disclosed methods, uses and kits, the IL-17 antibody or antigen-binding fragment thereof (e.g., secukinumab) is disposed in a pharmaceutical formulation, wherein said pharmaceutical formulation further comprises a buffer and a stabilizer.

In preferred embodiments of the disclosed methods, uses and kits, the pharmaceutical formulation is a liquid pharmaceutical formulation.

In preferred embodiments of the disclosed methods, uses and kits, the pharmaceutical formulation is a lyophilized pharmaceutical formulation.

In preferred embodiments of the disclosed methods, uses and kits, the pharmaceutical formulation is disposed within at least one pre-filled syringe, at least one vial, at least one injection pen, or at least one autoinjector.

In preferred embodiments of the disclosed methods, uses and kits, the at least one pre-filled syringe, at least one vial, at least one injection pen, or at least one autoinjector is disposed within a kit, and wherein said kit further comprises instructions for use.

In preferred embodiments of the disclosed methods, uses and kits, the dose of the IL-17 antibody or antigen-binding fragment thereof (e.g., secukinumab) is 300 mg, which is administered to the patient as a single subcutaneous administration in a total volume of 2 milliliters (mL) from a formulation comprising 150 mg/ml of the IL-17 antibody or antigen-binding fragment thereof (e.g., secukinumab), wherein the pharmacological exposure of the patient to the IL-17 antibody or antigen-binding fragment (e.g., secukinumab) is equivalent to the pharmacological exposure of the patient to the IL-17 antibody or antigen-binding fragment thereof (e.g., secukinumab) using two separate subcutaneous administrations of a total volume of 1 ml each of the same formulation.

In preferred embodiments of the disclosed methods, uses and kits, the dose of the IL-17 antibody or antigen-binding fragment thereof (e.g., secukinumab) administered to the patient is 300 mg, which is administered as two separate subcutaneous administrations in a volume of 1 mL each from a formulation comprising 150 mg/ml of the IL-17 antibody or antigen-binding fragment thereof (e.g., secukinumab).

In preferred embodiments of the disclosure, when said method is used to treat a population of patients having LN, at least 50% of said patients achieve a daily steroid dose of ≤10 mg/day following a steroid tapering regimen during treatment with the IL-17 antibody or antigen-binding fragment thereof (e.g., secukinumab).

In preferred embodiments of the disclosure, when said method is used to treat a population of patients having LN, at least 50% of said patients achieve a daily steroid dose of ≤5 mg/day following a steroid tapering regimen during treatment with the IL-17 antibody or antigen-binding fragment thereof (e.g., secukinumab).

In preferred embodiments of the disclosure, when said method is used to treat a population of patients having LN, at least 15% of said patients achieve a CRR following 52 weeks of treatment with the IL-17 antibody or antigen-binding fragment thereof (e.g., secukinumab).

In preferred embodiments of the disclosure, when said method is used to treat a population of patients having LN, at least 20% of said patients achieve a CRR following 52 weeks of treatment with the IL-17 antibody or antigen-binding fragment thereof (e.g., secukinumab).

In preferred embodiments of the disclosed methods, uses and kits, the patient achieves an improvement in UPCR of ≥75% by week 52.

In preferred embodiments of the disclosed methods, uses and kits, the patient is treated with the IL-17 antibody or antigen-binding fragment thereof (e.g., secukinumab) for at least one year.

In preferred embodiments of the disclosure, the IL-17 antibody or antigen-binding fragment thereof (e.g., secukinumab) is a monoclonal antibody.

In preferred embodiments of the disclosure, the IL-17 antibody or antigen-binding fragment thereof (e.g., secukinumab) is a human or humanized antibody.

In preferred embodiments of the disclosure, the IL-17 antibody or antigen-binding fragment thereof (e.g., secukinumab) is a human antibody.

In preferred embodiments of the disclosed methods, uses and kits, the IL-17 antibody or antigen-binding fragment is a human monoclonal antibody.

In preferred embodiments of the disclosure, the IL-17 antibody or antigen-binding fragment thereof (e.g., secukinumab) is a human antibody of the IgG₁ subtype.

In preferred embodiments the IL-17 antibody or antigen-binding fragment thereof (e.g., secukinumab) has a kappa light chain.

In preferred embodiments of the disclosure, the IL-17 antibody or antigen-binding fragment thereof (e.g., secukinumab) is a human antibody of the IgG₁ kappa type.

In preferred embodiments of the disclosed methods, uses and kits, the IL-17 antibody or antigen-binding fragment (e.g., secukinumab) has a Tₘₐₓ of about 7-8 days.

In preferred embodiments of the disclosed methods, uses and kits, the IL-17 antibody or antigen-binding fragment thereof (e.g., secukinumab) has an absolute bioavailablilty of about 60% —about 80%.

In preferred embodiments of the disclosure, the IL-17 antibody or antigen-binding fragment thereof is secukinumab.

Disclosed herein are methods of treating an adult patient with active LN who previously had an inadequate response to prior treatment with standard-of-care LN therapy, comprising administering a dose of about 300 mg secukinumab subcutaneously to said patient during week 0, 1, 2, 3, and 4, and then every four weeks thereafter, and further comprising concomitantly administering to said patient standard-of-care LN therapy, wherein said patient has ISN/RPS Class III or IV LN.

Disclosed herein are methods of treating a patient (e.g., an adult patient) with active lupus nephritis, comprising administering a dose of about 300 mg secukinumab subcutaneously to said patient during week 0, 1, 2, 3, and 4, and then every four weeks thereafter, and further comprising concomitantly administering to said patient standard-of-care LN therapy.

Disclosed herein are methods of treating a patient (e.g., an adult patient) with active lupus nephritis, comprising administering a dose of about 300 mg secukinumab subcutaneously to said patient during week 0, 1, 2, 3, and 4, and then every four weeks thereafter, and further comprising concomitantly administering to said patient standard-of-care LN therapy, wherein said patient has ISN/RPS Class III or IV LN.

In some embodiments, the standard-of-care LN therapy comprises treatment with MPA or cyclophosphamide (CYC) and, optionally, a steroid.

Disclosed herein are methods of treating a patient (e.g., an adult patient) with active lupus nephritis, comprising administering a dose of about 300 mg secukinumab subcutaneously to said patient during week 0, 1, 2, 3, and 4, and then every four weeks thereafter.

Disclosed herein are methods of treating a patient (e.g., an adult patient) having LN, comprising intravenously (IV) administering to the patient a dose of about 6 mg/kg secukinumab once during week 0, and thereafter administering an IV dose of about 3 mg/kg secukinumab every four weeks, beginning during week 4.

Disclosed herein are methods of treating a patient (e.g., an adult patient) having active lupus nephritis, comprising intravenously (IV) administering to the patient a dose of about 4 mg/kg to about 9 mg/kg (preferably about 6 mg/kg) secukinumab once during week 0, and thereafter administering an IV dose of about 2 mg/kg to about 4 mg/kg (preferably about 3 mg/kg) secukinumab every four weeks, beginning during week 4.

### Kits

The disclosure also encompasses kits for treating LN. Such kits comprise an IL-17 antagonist, e.g., IL-17 binding molecule (e.g., IL-17 antibody or antigen-binding fragment thereof, e.g., secukinumab) or IL-17 receptor binding molecule (e.g., IL-17 antibody or antigen-binding fragment thereof) (e.g., in liquid or lyophilized form) or a pharmaceutical composition comprising the IL-17 antagonist (described supra). Additionally, such kits may comprise means for administering the IL-17 antagonist (e.g., an autoinjector, a syringe and vial, a prefilled syringe, a prefilled pen) and instructions for use. These kits may contain additional therapeutic HS agents (described supra) for treating LN, e.g., for delivery in combination with the enclosed IL-17 antagonist, e.g., IL-17 binding molecule, e.g., IL-17 antibody, e.g., secukinumab. Such kits may also comprise instructions for administration of the IL-17 antagonist (e.g., IL-17 antibody, e.g., secukinumab) to treat the LN patient. Such instructions may provide the dose (e.g., 3 mg/kg, 6 mg/kg, 300 mg, 450 mg), route of administration (e.g., IV, SC), and dosing regimen (e.g., weekly, monthly, weekly and then monthly, weekly and then every other week, etc.) for use with the enclosed IL-17 antagonist, e.g., IL-17 binding molecule, e.g., IL-17 antibody, e.g., secukinumab.

The phrase “means for administering” is used to indicate any available implement for systemically administering a drug to a patient, including, but not limited to, a pre-filled syringe, a vial and syringe, an injection pen, an autoinjector, an IV drip and bag, a pump, etc. With such items, a patient may self-administer the drug (i.e., administer the drug without the assistance of a physician) or a medical practitioner may administer the drug. In some embodiments, a total dose of 300 mg is to be delivered in a total volume of 2 ml, which is disposed in two PFSs or autoinjectors, each PFS or autoinjector containing a volume of 1 ml having 150 mg/ml of the IL-17 antibody, e.g., secukinumab. In this case, the patient receives two 1 ml injections (a multi-dose preparation). In preferred embodiments, a total dose of 300 mg is to be delivered in a total volume of 2 ml having 150 mg/ml of the IL-17 antibody, e.g., secukinumab, which is disposed in a single PFS or autoinjector. In this case, the patient receives one 2 ml injection (a single dose preparation).

Disclosed herein are kits for use treating a patient having LN, comprising an IL-17 antagonist (e.g., IL-17 binding molecule, e.g., IL-17 antibody or antigen-binding fragment thereof, e.g., secukinumab) and means for administering the IL-17 antagonist to the LN patient. In some embodiments, the kit further comprises instructions for administration of the IL-17 antagonist, wherein the instructions indicate that the IL-17 antagonist (e.g., IL-17 binding molecule, e.g., IL-17 antibody or antigen-binding fragment thereof, e.g., secukinumab) is to be administered to the patient SC at a dose of about 150 mg—about 300 mg (e.g., about 150 mg, about 300 mg) weekly during week 0, 1, 2, 3, and 4 and then every four weeks thereafter. In some embodiments, the kit further comprises instructions for administration of the IL-17 antagonist, wherein the instructions indicate that the IL-17 antagonist (e.g., IL-17 binding molecule, e.g., IL-17 antibody or antigen-binding fragment thereof, e.g., secukinumab) is to be administered to the patient intravenously (IV) at a dose of about 4 mg/kg—about 9 mg/kg (preferably about 6 mg/kg) once during week 0, and thereafter, as an IV dose of about 2—about 4 mg/kg (preferably about 3 mg/kg) every 4 weeks (monthly), beginning during week 4.

### General

In most preferred embodiments of the disclosed methods, kits, or uses, the IL-17 antibody or antigen-binding fragment thereof is secukinumab.

In preferred embodiments of the disclosed methods, kits, or uses, the dose size is flat (also referred to as a “fixed” dose, which differs from weight-based or body surface area-based dosing), the dose is 300 mg, the route of administration is SC, and the regimen is administration at week 0, 1, 2, 3, 4, 8, 12 etc. (weekly during week 0, 1, 2, 3, and 4, and then every four weeks, beginning during week 8) or administration at week 0, 1, 2, 3, 4, 6, 8, 10, 12 etc. (weekly during week 0, 1, 2, 3, and 4, and then every other week, beginning during week 6).

In other embodiments of the disclosed methods, kits, or uses, the dose size is weight-based, the single induction dose is 6 mg/kg, the route of administration is IV, the maintenance dose is 3 mg/kg, and the regimen is administration at week 0 (induction), 4, 8, 12, 16, 20, et.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural references unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference. The following Examples are presented in order to more fully illustrate the preferred embodiments of the disclosure. These examples should in no way be construed as limiting the scope of the disclosed subject matter, as defined by the appended claims.

### EXAMPLES

### Example 1

A two-year, phase III randomized, double-blind, parallel-group, placebo-controlled trial to evaluate the safety, efficacy, and tolerability of 300 mg s.c. secukinumab versus placebo, in combination with SoC therapy, in patients with active lupus nephritis.

### Study Purpose

The purpose of this trial is to evaluate the efficacy and safety of subcutaneous (SC) secukinumab 300 mg compared to placebo, in combination with standard of care therapy (SoC), in subjects with active lupus nephritis (ISN/RPS Class III or IV, with or without co-existing class V features).

Background SoC will consist of induction therapy with mycophenolic acid (MPA) (which refers to Mycophenolate mofetil (MMF) (Cellcept® or generic equivalent), or enteric-coated MPA sodium (Myfortic® or generic equivalent) at equivalent doses (oral), or Cyclophosphamide (CYC) (i.v.), followed by maintenance therapy with MPA. In addition, all subjects will receive i.v. and/or oral corticosteroids.

### Study Design

This is a pivotal, randomized, double-blind, placebo controlled trial evaluating at Week 52 the efficacy and safety of secukinumab versus placebo in subjects with active LN also receiving background SoC regimen. Long-term efficacy, safety and tolerability will be collected up to 2 years.

The SoC regimen will consist of induction therapy with MPA or CYC, followed by maintenance therapy with MPA. The choice of background SoC induction therapy will be at investigator's discretion. At Randomization, subjects will be stratified on the basis of the SoC induction therapy they will receive during the study, MPA or CYC-based, to ensure a balanced representation in each of the treatment arms (secukinumab or placebo). The target will be to have a maximum of 25% of randomized subjects receiving CYC-based induction therapy.

In addition, steroids will be administered through i.v. pulses followed by oral daily doses.

The primary endpoint analysis will be performed after all subjects have completed the visit associated with the primary endpoint (Week 52).

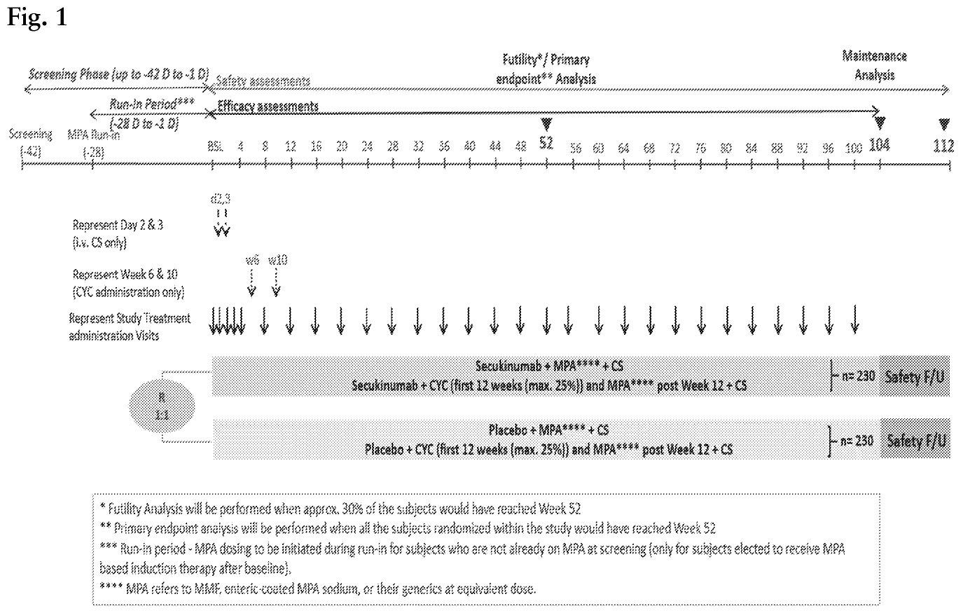

The study design is shown in FIG. 1, and consists of the following parts:
a. Screening (up to 42 days/6 weeks)
b. Run-in period (optional): For subjects who will receive MPA as SoC induction therapy as per investigator's decision and who are not already on MPA at Screening, MPA dosing will be initiated during a run-in period before Randomization (for up to 4 weeks prior to the first dose of secukinumab)
c. Treatment Period: Duration of 104 weeks of treatment with secukinumab/placebo in addition to SoC treatment (with last dose given at Week 100)
d. Follow-up period: Duration of 8 weeks (last visit performed 12 weeks after last dose of study medication)

### Rationale for Dose and Regimen

Secukinumab dosing will start with initial dosing of 300 mg s.c. injections at Baseline, Weeks 1, 2, 3, and 4, followed by dosing every 4 weeks. This dosing regimen is approved for treatment of other autoimmune diseases (PsO, PsA). Our data strongly suggests that secukinumab operates at the plateau of the dose-exposure-response curve in these autoimmune diseases, which is one of the reasons to select this dose level in LN as well. Initial weekly dosing during the first month is also expected to enable rapid achievement of effective drug concentrations, and lead to a more rapid onset of clinical response.

Nevertheless, it has to be noted that due to kidney damage, proteinuria is commonly observed in patients with LN. The effect of renal impairment on the PK of biologics is dependent on the ability of the compound to undergo glomerular filtration, which is largely driven by molecular weight (MW). Secukinumab has a MW of ca. 148 kDa, and renal clearance usually plays a minimal role in the elimination of biologics with MW greater than 69 kDa (Meibohm (2012) J. Clin. Pharm. 52(1):545-625). An association between increased Baseline proteinuria and increased clearance was observed in the population PK analysis of belimumab (a human mAb that inhibits B-cell activating factor, BAFF) in SLE (Struemper et al (2013) J. Clin. Pharm. 53(7):711-20). Also, there is evidence that in some forms of renal disease, such as diabetic nephropathy, there may be an increase in the renal elimination of IgGs (Bakoush et al. (2002) Kidney International 61:203-8). However, minor changes in distribution volume or increased clearance of secukinumab in LN patients should not dramatically change the PK characteristics of the drug.

A summary table follows:

| | |
|---|---|
| Full Title | A two-year, phase III randomized, double-blind, parallel-group, placebo-controlled |
| | trial to evaluate the safety, efficacy, and tolerability of 300 mg s.c. secukinumab |
| | versus placebo, in combination with SoC therapy, in patients with active lupus |
| | nephritis |
| Purpose and | The purpose of this trial is to evaluate the efficacy and safety of subcutaneous |
| rationale | secukinumab 300 mg compared to placebo, in combination with standard of care |
| | therapy (SoC), in subjects with active lupus nephritis (ISN/RPS Class III or IV, with |
| | or without co-existing class V features). Background SoC will consist of induction |
| | therapy with mycophenolic acid (MPA) (which refers to Mycophenolate mofetil |
| | (MMF) (Cellcept ® or generic equivalent), or enteric-coated MPA sodium (Myfortic ® or |
| | generic equivalent) at equivalent doses (oral), or Cyclophosphamide (CYC) |
| | (i.v.), followed by maintenance therapy with MPA (MMF, enteric-coated MPA |
| | sodium, or their generics). In addition, all subjects will receive i.v. and/or oral |
| | corticosteroids. |
| | The aim of the study is to demonstrate the efficacy and safety of secukinumab in LN |
| | that will enable registration for the indication of lupus nephritis. |
| Primary | The primary objective is to demonstrate that secukinumab 300 mg is superior to |
| Objective(s) | placebo in Complete Renal Response (CRR) rate at Week 52 in active lupus |
| | nephritis (ISN/RPS Class III or IV, with or without co-existing Class V features) |
| | subjects on a background of SoC therapy |
| Secondary | Objective 1: To demonstrate superiority of secukinumab compared to placebo in |
| Objectives | change from Baseline in 24-hour UPCR at Week 52 |
| | Objective 2: To demonstrate superiority of secukinumab compared to placebo in |
| | proportion of subjects achieving partial renal response (PRR) at Week 52 |
| | Objective 3: To demonstrate superiority of secukinumab compared to placebo in |
| | average daily dose of oral corticosteroids administered between Week 16 and Week |
| | 52 |
| | Objective 4: To demonstrate superiority of secukinumab compared to placebo in |
| | proportion of subjects achieving PRR at Week 24 |
| | Objective 5: To demonstrate superiority of secukinumab compared to placebo in time |
| | to achieve CRR |
| | Objective 6: To demonstrate superiority of secukinumab compared to placebo in time |
| | to achieve PRR |
| | Objective 7: To demonstrate superiority of secukinumab compared to placebo in time |
| | to achieve first morning void Urine Protein-to-Creatinine Ratio (UPCR) ≤0.5 mg/mg |
| | Objective 8: To demonstrate superiority of secukinumab compared to placebo in |
| | change in Functional Assessment of Chronic Illness Therapy - Fatigue (FACIT- |
| | Fatigue ©) score at Week 52 |
| | Objective 9: To demonstrate superiority of secukinumab compared to placebo in |
| | patient's health related quality of life via Medical Outcome Short Form Health Survey |
| | (SF-36 Physical Component Summary (PCS)) score at Week 52 |
| | Objective 10: To demonstrate superiority of secukinumab compared to placebo in |
| | change of LupusQoL (Physical Health) score at Week 52 |
| | Objective 11: To evaluate the safety and tolerability of secukinumab s.c. as an add- |
| | on therapy to Standard of Care in lupus nephritis subjects |
| | Objective 12: To estimate the proportion of subjects with maintained renal response |
| | at Week 104 |
| | Objective 13: To estimate the proportion of subjects with improved or maintained renal |
| | response at Week 104 |
| Study design | This is a pivotal, randomized, double-blind, placebo controlled trial evaluating at Week |
| | 52 the efficacy and safety of secukinumab versus placebo in subjects with active lupus |
| | nephritis also receiving background SoC regimen. In addition, long-term efficacy, |
| | safety and tolerability will be collected up to 2 years. |
| Population | The study population will be comprised of adult male and female subjects in the age |
| | range of 18-75 years with a renal biopsy (results current or within the 6 months prior |
| | to Screening) showing active glomerulonephritis WHO or ISN/RPS Class III or IV LN |
| | [excluding III (C), IV-S (C) and IV-G (C)], with or without co-existing class V features, |
| | who are inadequately controlled with previous SoC defined as having UPCR ≥1 and |
| | active urinary sediment (presence of cellular casts which are granular casts or red |
| | blood cells) or hematuria (>5 red blood cells per high power field)). |
| | At randomization, subjects will be stratified on the basis of the SoC induction therapy |
| | they will receive during the study, MPA or CYC-based, to ensure a balanced |
| | representation in each of the treatment arms (secukinumab or placebo). The target |
| | will be to have a maximum of 25% of randomized subjects receiving CYC-based |
| | induction therapy. |
| Key Inclusion | Subjects eligible for inclusion in this study must meet all of the following criteria: |
| criteria | 1. Adult male and female subjects aged 18-75 years old at the time of Baseline |
| | 2. Confirmed diagnosis of: |
| | SLE with documented history of at least 4 of the 11 criteria for SLE as defined by |
| | the American College of Rheumatology (ACR). [NOTE: The 4 criteria do not have to |
| | be present at the time of Screening], |
| | OR |
| | Lupus nephritis as the sole clinical criterion in the presence of ANA or anti-dsDNA |
| | antibodies. |
| | 3. Active lupus nephritis, as defined by meeting the 4 following criteria: |
| | Biopsy within 6 months prior to Screening visit indicating active glomerulonephritis |
| | WHO or ISN/RPS Class III or IV LN [excluding III (C), IV-S (C) and IV-G (C)]; subjects |
| | are permitted to have co-existing Class V. If no biopsy was performed within 6 months |
| | of Screening, a biopsy will need to be performed during the Screening period, after all |
| | other inclusion/exclusion criteria would have been verified. |
| | UPCR ≥1 at Screening |
| | Estimated eGFR >30 mL/min/1.73 m² by Chronic Kidney Disease Epidemiology |
| | Collaboration (CKD-EPI) |
| | Active urinary sediment (presence of cellular casts (granular or red blood cell casts) |
| | or hematuria (>5 red blood cells per high power field)) |
| | 4. Subjects must be currently on, or willing to initiate SoC induction therapy for LN |
| | according to the institutional practices using MPA (MMF or enteric-coated MPA |
| | sodium) or low-dose CYC in addition to corticosteroids. |
| | 5. If the subject is on cholesterol-lowering agents, the dose must be stable for at least |
| | 7 days prior to Randomization. |
| | 6. Subjects must be treated with anti-malarials (e.g., hydroxychloroquine), unless |
| | contra-indicated, and the dose must be stable for at least 10 days prior to |
| | Randomization. |
| | 7. Able to provide signed informed consent. |
| Key Exclusion | Subjects meeting any of the following criteria are not eligible for inclusion in this |
| criteria | study. |
| | 1. Severe renal impairment as defined by i.) Stage 4 CKD, or ii.) presence of oliguria |
| | (defined as a documented urine volume <400 mL/24 hrs), or iii.) ESRD requiring |
| | dialysis or transplantation |
| | 2. Known intolerance/hypersensitivity to MPA (MMF or enteric-coated MPA sodium), |
| | or oral corticosteroids, or any component of the study treatment |
| | 3. Subjects having received any other biologic immunomodulatory therapy within 6 |
| | months prior to Screening, excluding belimumab where 3 months are acceptable |
| | 4. Previous exposure to secukinumab (AIN457) or any other biologic drug targeting |
| | IL-17 or the IL-17 receptor |
| | 5. Subjects having received any investigational drug within 1 month or five times the |
| | half-life, whichever is longer |
| | 6. Receipt of more than 3000 mg i.v. pulse methylprednisolone (cumulative dose) |
| | within the 12 weeks prior to Baseline |
| | 7. Treatment with a systemic calcineurin inhibitor (e.g., cyclosporine, tacrolimus) |
| | within 12 weeks prior to Baseline |
| | 8. CYC use (i.v. or oral) within the month prior to Baseline |
| | 9. Subjects requiring dialysis within the previous 12 months before Screening |
| | 10. History of renal transplant |
| | 11. Any severe progressive or uncontrolled concurrent medical condition, including |
| | recent severe thromboembolic events, that, in the opinion of the principal investigator, |
| | renders the subject unsuitable for the trial |
| | 12. Active ongoing inflammatory diseases that might confound the evaluation of the |
| | benefit of secukinumab therapy, including inflammatory bowel disease |
| | 13. Presence of investigator-identified significant medical problems which at the |
| | investigator's discretion will prevent the subject from participating in the study, |
| | including but not limited to the following: myocarditis, pericarditis, poorly controlled |
| | seizure disorder, acute confusional state, depression, severe manifestations of |
| | neuropsychiatric SLE (NPSLE) |
| | 14. Chest X-ray, computerized tomography (CT) scan, or MRI with evidence of |
| | ongoing infectious or malignant process, obtained within 12 weeks prior to |
| | Randomization and evaluated by a qualified physician |
| | 15. History of chronic, recurrent systemic infections, active tuberculosis infection, or |
| | active systemic infections during the last two weeks (exception: common cold) prior |
| | to Randomization |
| | 16. Known infection with human immunodeficiency virus (HIV), hepatitis B or hepatitis |
| | C at Screening or Randomization |
| | 17. History of lymphoproliferative disease or any known malignancy or history of |
| | malignancy of any organ system treated or untreated within the past 5 years, |
| | regardless of whether there is evidence of local recurrence or metastases (except for |
| | skin Bowen's disease or basal cell carcinoma or actinic keratoses that have been |
| | treated with no evidence of recurrence in the past 12 weeks, carcinoma in situ of the |
| | cervix or non-invasive malignant colon polyps that have been removed) |
| | 18. Any of the following abnormal laboratory values on Screening evaluations as |
| | reported by Central Laboratory: |
| | Aspartate aminotransferase (AST), alanine aminotransferase (ALT), or amylase >2.5 × ULN |
| | Hemoglobin <8 g/dL |
| | Neutrophils <1.0 × 10⁹/L |
| | Platelet count <50 × 10⁹/L |
| | 19. Inability or unwillingness to undergo repeated venipuncture (e.g., because of poor |
| | tolerability or lack of venous access) |
| | 20. History or evidence of ongoing alcohol or drug abuse, within the last six months |
| | before Randomization |
| | 21. Pregnant or lactating women |
| | 22. Women of childbearing potential, defined as all women physiologically capable of |
| | becoming pregnant, unless they are using highly effective methods of contraception |
| | during the entire study or longer if required by locally approved prescribing information |
| | (e.g., in European Union (EU) 20 weeks) |
| Study | At Baseline, all eligible subjects will be randomized to one of the two treatment arms |
| treatment | in a 1:1 ratio via Interactive Response Technology (IRT): |
| | Arm 1: LN subjects will receive secukinumab 300 mg s.c. (2 × 1.0 mL PFS of 150 |
| | mg dose) at Randomization, Weeks 1, 2 and 3, and every 4 weeks from Week 4 |
| | until week 100 |
| | Arm 2: LN subjects will receive placebo s.c. (2 × 1.0 mL PFS of 0 mg dose) at |
| | Randomization, Weeks 1, 2 and 3, and every 4 weeks from Week 4 until week |
| | 100 |
| | At Randomization, subjects will be stratified on the basis of the SoC induction therapy |
| | they will receive during the study, MPA or CYC-based, to ensure a balanced |
| | representation in each of the treatment arms (secukinumab or placebo). |
| Efficacy | Assessment of CRR, defined as eGFR within the normal range or no less than |
| assessments | 85% of Baseline AND 24-hour UPCR ≤0.5 mg/mg |
| | Time to achieve UPCR ≤0.5 mg/mg |
| | Assessment of PRR, defined as ≥50% reduction in 24-hour UPCR to sub- |
| | nephrotic levels AND normal eGFR or no less than 85% of Baseline |
| | Average daily dose of oral corticosteroids |
| | Time to achieve CRR |
| | Time to achieve PRR |
| | FACIT-Fatigue © score |
| | SF-36 PCS score |
| | LupusQoL Physical Health score |
| Key safety | Physical examinations |
| assessments | Vital signs |
| | Height and weight |
| | Laboratory evaluations (hematology, clinical chemistry, coagulation panel, local |
| | urinalysis, 24-hour urine collection, lipid panel, autoantibodies, selected serum |
| | complement components, circulating immunoglobulins (Igs) and pregnancy test) |
| | Chest X-ray |
| | Evaluation of AEs and SAEs |
| Other | Assessment of Urine Albumin-to-Creatinine Ratio (UACR) |
| assessments | Evaluation of renal proteinuric flare, defined as a persistent increase in the first |
| | morning void UPCR >1.0 mg/mg after CRR is achieved OR a doubling of |
| | proteinuria, in first morning void UPCR with values >1.0 mg/mg after a PRR is |
| | achieved |
| | Inactive urinary sediments |
| | Clinician reported outcomes (CROs): SLEDAI-2000, CLASI, DAS28-CRP, |
| | LLDAS |
| | Progression in CKD or to ESRD |
| | PK: secukinumab concentrations |
| | Immunogenicity |
| | Biomarkers (urine and serum) |
| | Pharmacogenetics; DNA and RNA analysis |
| Data analysis | The primary efficacy endpoint is the CRR at Week 52. |
| | The statistical hypothesis tested for the primary objective is that there is no difference |
| | in the proportion of subjects fulfilling the response criteria at Week 52 between the |
| | secukinumab regimen and placebo regimens. |
| | Let pⱼ denote the proportion of responders at Week 52 for treatment regimens j, j = 0, |
| | 1 where |
| | 0 corresponds to placebo regimen, |
| | 1 corresponds to secukinumab, |
| | In statistical terms, H1: p1 = p0, HA1: p1 ≠ p0, i.e., |
| | H1: secukinumab is not different to placebo regimen with respect to CRR at Week 52 |
| | Logistic regression model adjusting for SoC, race and Baseline UPCR will be used |
| | for the primary analysis. Difference in marginal response proportions with p-value and |
| | respective 95% confidence interval will be estimated from the logistic regression |
| | model. |
| | Safety analyses will include summaries of AEs, laboratory measurements, and vital |
| | signs. |
| | Full details of all data analyses will be specified in statistical analysis plan. |

What is claimed is:

1. A method of treating lupus nephritis (LN), comprising subcutaneously (SC) administering to a patient in need thereof a dose of about 150 mg of an IL-17 antibody, or an antigen-binding fragment thereof, weekly during weeks 0, 1, 2, 3, and 4, and every four weeks thereafter, wherein the IL-17 antibody or antigen-binding fragment thereof comprises:
i) an immunoglobulin variable heavy (V_{H}) domain comprising the amino acid sequence set forth as SEQ ID NO:8 and an immunoglobulin variable light (V_{L}) domain comprising the amino acid sequence set forth as SEQ ID NO:10;
ii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or
iii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6.

2. A method of treating lupus nephritis (LN), comprising subcutaneously (SC) administering to a patient in need thereof a dose of about 300 mg of an IL-17 antibody, or an antigen-binding fragment thereof, weekly during weeks 0, 1, 2, 3, and 4, and every four weeks thereafter, wherein the IL-17 antibody or antigen-binding fragment thereof comprises:
i) an immunoglobulin variable heavy (V_{H}) domain comprising the amino acid sequence set forth as SEQ ID NO:8 and an immunoglobulin variable light (V_{L}) domain comprising the amino acid sequence set forth as SEQ ID NO:10;
ii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or
iii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6.

3. A method of treating LN, comprising intravenously (IV) administering to a patient in need thereof a dose of about 4 mg/kg—about 9 mg/kg (preferably about 6 mg/kg) of an IL-17 antibody, or an antigen-binding fragment thereof, once during week 0, and thereafter administering an IV dose of about 2 mg/kg—about 4 mg/kg (preferably about 3 mg/kg) of the IL-17 antibody, or an antigen-binding fragment thereof every four weeks, beginning during week four, wherein the IL-17 antibody or antigen-binding fragment thereof comprises:
i) an immunoglobulin variable heavy (V_{H}) domain comprising the amino acid sequence set forth as SEQ ID NO:8 and an immunoglobulin variable light (V_{L}) domain comprising the amino acid sequence set forth as SEQ ID NO:10;
ii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or
iii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6.

4. The method according to any of claims 1-3, wherein the IL-17 antibody or antigen-binding fragment thereof binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 antibody has a K_{D} of about 100-200 pM as measured by a biosensor system, and wherein the IL-17 antibody has an in vivo half-life of about 23 to about 30 days.

5. The method according to any of the above claims, wherein prior to treatment with the IL-17 antibody or antigen-binding fragment thereof, the patient was administered mycophenolic acid (MPA) or cyclophosphamide (CYC), and, optionally at least one steroid.

6. The method according to claim 5, wherein prior to treatment with the IL-17 antibody or antigen-binding fragment thereof, the LN was inadequately controlled by the prior treatment with MPA or CYC, and, optionally the at least one steroid.

7. The method according to any of the above claims, wherein during treatment with the IL-17 antibody or antigen-binding fragment thereof, the patient is concomitantly administered MPA or CYC, and, optionally at least one steroid.

8. The method according to claim 7, wherein during treatment with the IL-17 antibody or antigen-binding fragment thereof, the dose of MPA or CYC administered to the patient is reduced, and wherein the patient does not experience a flare as a result of said reduction.

9. The method according to claim 7 or 8, wherein during treatment with the IL-17 antibody or antigen-binding fragment thereof, the dose of the at least one steroid administered to the patient is reduced using a taper regimen, and wherein the patient does not experience a flare as a result of said reduction.

10. The method according to any of the above claims, wherein the patient does not have concomitant plaque-type psoriasis.

11. The method according to any of the above claims, wherein the patient has active LN.

12. The method according to any of the above claims, wherein the patient has International Society of Nephrology/Renal Pathology Society (ISN/RPS) Class III or IV LN.

13. The method according to claim 12, wherein the ISN/RPS Class III IN is not Class III(C).

14. The method according to claim 12, wherein the ISN/RPS Class IV LN is not Class IV—S(C) or IV-G(C).

15. The method according to any of the above claims, wherein the patient has features of ISN/RPS Class V LN.

16. The method according to any of the above claims, wherein said patient achieves a complete renal response (CRR) after one year of treatment

17. The method according to any of the above claims, wherein said patient achieves a partial renal response (PRR) after one year of treatment.

18. The method according to any of the above claims, wherein the patient is additionally administered at least one LN agent selected from the group consisting of rituximab, ocrelizumab, abatacept, azathioprine, a calcineurin inhibitor, cyclosporine A, tacrolimus, cyclophosphamide, mycophenolic acid, voclosporin, belimumab, ustekinumab, iguratimod, anifrolumab, BI655064, CFZ533, and combinations thereof.

19. The method according to any of the above claims, wherein the patient is an adult.

20. The method according to any of the above claims, wherein the IL-17 antibody or antigen-binding fragment thereof is disposed in a pharmaceutical formulation, wherein said pharmaceutical formulation further comprises a buffer and a stabilizer.

21. The method according to claim 20, wherein the pharmaceutical formulation is a liquid pharmaceutical formulation.

22. The method according to claim 20, wherein the pharmaceutical formulation is a lyophilized pharmaceutical formulation.

23. The method according to any of claims 20-22, wherein the pharmaceutical formulation is disposed within at least one pre-filled syringe, at least one vial, at least one injection pen, or at least one autoinjector.

24. The method according to claim 23, wherein the at least one pre-filled syringe, at least one vial, at least one injection pen, or at least one autoinjector is disposed within a kit, and wherein said kit further comprises instructions for use.

25. The method according to any of claim 2 or 4-24, wherein the dose of the IL-17 antibody or antigen-binding fragment thereof is 300 mg, which is administered to the patient as a single subcutaneous administration in a total volume of 2 milliliters (mL) from a formulation comprising 150 mg/ml of the IL-17 antibody or antigen-binding fragment thereof, wherein the pharmacological exposure of the patient to the IL-17 antibody or antigen-binding fragment is equivalent to the pharmacological exposure of the patient to the IL-17 antibody or antigen-binding fragment thereof using two separate subcutaneous administrations of a total volume of 1 ml each of the same formulation.

26. The method according to any of claim 2 or 4-24, wherein the dose of the IL-17 antibody or antigen-binding fragment thereof administered to the patient is 300 mg, which is administered as two separate subcutaneous administrations in a volume of 1 mL each from a formulation comprising 150 mg/ml of the IL-17 antibody or antigen-binding fragment

27. The method according to any of the above claims, wherein the IL-17 antibody or antigen-binding fragment thereof has a Tₘₐₓ of about 7-8 days.

28. The method according to any of the above claims, wherein the IL-17 antibody or antigen-binding fragment thereof has an absolute bioavailablilty of about 60%—about 80%.

29. The method according to any of the above claims, wherein the IL-17 antibody or antigen-binding fragment thereof is a human monoclonal antibody.

30. The method according to any of the above claims, wherein the IL-17 antibody or antigen-binding fragment thereof is of the IgG₁/kappa isotype.

31. The method according to any of the above claims, wherein, when said method is used to treat a population of patients having LN, at least 50% of said patients achieve a daily steroid dose of ≤10 mg/day following a steroid tapering regimen during treatment with the IL-17 antibody or antigen-binding fragment thereof.

32. The method according to any of the above claims, wherein, when said method is used to treat a population of patients having LN, at least 50% of said patients achieve a daily steroid dose of ≤5 mg/day following a steroid tapering regimen during treatment with the IL-17 antibody or antigen-binding fragment thereof.

33. The method according to any of the above claims, wherein, when said method is used to treat a population of patients having LN, at least 15% of said patients achieve a CRR following 52 weeks of treatment with the IL-17 antibody or antigen-binding fragment thereof.

34. The method according to any of the above claims, wherein, when said method is used to treat a population of patients having LN, at least 20% of said patients achieve a CRR following 52 weeks of treatment with the IL-17 antibody or antigen-binding fragment thereof.

35. The method according to any of the above claims, wherein the patient achieves an improvement in UPCR of ≥75% by week 52.

36. The method according to any of the above claims, wherein the patient is treated with the IL-17 antibody or antigen-binding fragment thereof for at least one year.

37. The method according to any of the above claims, wherein the IL-17 antibody or antigen-binding fragment thereof is secukinumab.

38. A method of treating an adult patient with active LN who previously had an inadequate response to prior treatment with standard-of-care LN therapy, comprising administering a dose of about 300 mg secukinumab subcutaneously to said patient during week 0, 1, 2, 3, and 4, and then every four weeks thereafter, and further comprising concomitantly administering to said patient standard-of-care LN therapy, wherein said patient has ISN/RPS Class III or IV LN.

39. A method of treating a patient (e.g., an adult patient) with active lupus nephritis, comprising administering a dose of about 300 mg secukinumab subcutaneously to said patient during week 0, 1, 2, 3, and 4, and then every four weeks thereafter, and further comprising concomitantly administering to said patient standard-of-care LN therapy.

40. A method of treating a patient (e.g., an adult patient) with active lupus nephritis, comprising administering a dose of about 300 mg secukinumab subcutaneously to said patient during week 0, 1, 2, 3, and 4, and then every four weeks thereafter, and further comprising concomitantly administering to said patient standard-of-care LN therapy, wherein said patient has ISN/RPS Class III or IV LN.

41. The method of any one of claims 38-40, wherein said standard-of-care LN therapy comprises treatment with MPA or cyclophosphamide (CYC) and, optionally, a steroid.

42. A method of treating a patient (e.g., an adult patient) with active lupus nephritis, comprising administering a dose of about 300 mg secukinumab subcutaneously to said patient during week 0, 1, 2, 3, and 4, and then every four weeks thereafter.

43. A method of treating a patient (e.g., an adult patient) having LN, comprising intravenously (IV) administering to the patient a dose of about 6 mg/kg secukinumab once during week 0, and thereafter administering an IV dose of about 3 mg/kg secukinumab every four weeks, beginning during week 4.

44. A method of treating a patient (e.g., an adult patient) having active lupus nephritis, comprising intravenously (IV) administering to the patient a dose of about 4 mg/kg to about 9 mg/kg (preferably about 6 mg/kg) secukinumab once during week 0, and thereafter administering an IV dose of about 2 mg/kg to about 4 mg/kg (preferably about 3 mg/kg) secukinumab every four weeks, beginning during week 4.
